(19) United States
(12) Patent Application Publication
(10) Pub. No.: US 2006/0128694 A1
(43) Pub. Date: Jun. 15, 2006

(54) Adenosine A2a receptor antagonists for the treatment of extra-pyramidal syndrome and other movement disorders

(76) Inventors: Michael Grzelak, Wayne, NJ (US); John Hunter, Warren, NJ (US); Annamarie Pond, Biddeford, ME (US); Geoffrey Varty, Berkeley Heights, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/249,796

(22) Filed: Oct. 13, 2005

Related U.S. Application Data
(63) Continuation-in-part of application No. 11/234,644, filed on Sep. 23, 2005.
Application No. 11/234,644 is a continuation-in-part of application No. 10/738,906, filed on Dec. 17, 2003.
(60) Provisional application No. 60/435,321, filed on Dec. 19, 2002.

(51) Int. Cl. A61K 31/551; A61K 31/519; A61K 31/522

(52) U.S. Cl. 514/220; 514/252.16; 514/263.34; 514/263.32; 514/282; 514/259.41

(57) ABSTRACT

There is disclosed a method for the treatment or prevention of Extra Pyramidal syndrome (EPS), dystonia, restless legs syndrome (RLS) or periodic leg movement in sleep (PLMS) comprising the administration of an adenosine A2a receptor antagonist, alone or in combination with other agents useful for treating EPS, dystonia, RLS or PLMS; also claimed are pharmaceutical compositions consisting of an adenosine A2a receptor antagonist in combination with an antipsychotic agent, an anticonvulsant agent, lithium or an opioid.

### CROSS REFERENCE TO RELATED APPLICATIONS

This application is a continuation-in-part of U.S. Ser. No. ______, filed Sep. 23, 2005, which is a continuation-in-part of U.S. Ser. No. 10/738,906, filed Dec. 17, 2003, which claims the benefit of U.S. Provisional Application 60/435,321, filed Dec. 19, 2002.

### FIELD OF THE INVENTION

The present invention relates to the use of adenosine A₂ₐ receptor antagonists for the treatment of a variety of neurological syndromes involving the extra-pyramidal motor system (i.e. Extra-Pyramidal Syndrome) that occur following the acute and chronic use of almost all antipsychotic drugs. The invention also relates to the use of adenosine A₂ₐ receptor antagonists for the treatment of other abnormal movement disorders such as restless legs syndrome (RLS) and periodic limb movement in sleep (PLMS). The invention also relates to pharmaceutical compositions consisting of an adenosine A₂ₐ receptor antagonist and an antipsychotic agent for treating EPS, and to pharmaceutical compositions consisting of an A₂ₐ receptor antagonist and another agent useful for treating other abnormal movement disorders such as RLS or PLMS.

### BACKGROUND OF THE INVENTION

Extra-Pyramidal Syndrome (EPS) is a collective term for a series of adverse neurological reactions associated with the use of antipsychotic drugs. There are six different categories of EPS-related neurological syndromes of which four, dystonia, akathisia, pseudoparkinsonism (parkinsonian syndrome), and tardive dyskinesia, are particularly prevalent in patients taking antipsychotic medication. Dystonia is a painful spasm of the muscle groups of, in particular, the neck, jaw, back, pharynx, and larynx. It is most common in young males being treated with antipsychotic drugs, but can also be associated with the use of cocaine, tricyclic antidepressants, lithium and anticonvulsants such as phenytoin and carbamazepine. Pseudoparkinsonism manifests itself as akinesia (rigidity, stiffness and slow voluntary motion, stooped, shuffling walk) and tremor and these symptoms develop within weeks or months after initiation of therapy. Akathisia manifests itself as strong, subjective inner feelings of distress or discomfort characterized by motor restlessness. Often mistaken for agitation or anxiety, this common syndrome is frequently under-diagnosed and is the least responsive to treatment. Tardive dyskinesia is a late-appearing syndrome associated with chronic use of neuroleptic drugs. It occurs more frequently in older patients and is characterized by stereotypical, repetitive, involuntary, quick choreiform movements of the face, eyelids, mouth, tongue, extremities and trunk.

EPS is more prevalent with the use of typical antipsychotic agents but has also been reported with the use of atypical agents. Typical antipsychotics include loxapine, haloperidol, chlorpromazine, prochlorperazine and thiothixene. Atypical antipsychotics include clozapine, olanzapine, loxapine, quetiapine, ziprasidone, risperidone, aripiprazole, sertindole and zotepine.

Akathisia is also a characteristic of RLS and PLMS, as well as PLMD (periodic leg (or limb) movement disorder). RLS is a common disorder that causes patients to have an irresistible and unpleasant desire to move their legs; it usually manifests during periods of inactivity and/or at night, and can disturb sleep. Patients who do not have the typical RLS symptoms, but who do exhibit periodic leg movements that adversely impact sleep, are diagnosed with PLMS. Treatments for RLS and PLMS have included levodopa/carbidopa, levodopa/benserazide, dopamine agonists such as pramipexole and ropinerole, benzodiazepines, opioids, anticonvulsants and iron (ferrous sulfate). RLS and PLMS have been extensively described in the literature, for example by Saletu et al, Neuropsychobiology, 41, 4 (2000), p. 190-9.

The purine nucleotide, adenosine, is known to be an endogenous modulator of a number of physiological functions in the central (CNS) and peripheral nervous systems.

Adenosine exerts its biological actions through a class of membrane specific receptors which belong to the super family of receptors coupled with G proteins. Biochemical and pharmacological studies, together with advances in molecular biology, have allowed the identification of at least four subtypes of adenosine receptors: A₁, A₂ₐ, A_{2b} and A₃. Analogs of adenosine able to interact as antagonists with the A₁, A₂ₐ, A_{2b} and A₃ receptors have also been identified.

In the CNS, data has shown that A₂ₐ receptors are present in high density in the basal ganglia, known to be important in the control of fine motor movement. Moreover, selective antagonists for the A₂ₐ receptor are of pharmacological interest because of their demonstrated efficacy in reducing motor impairment thereby improving function in neurodegenerative diseases such as Parkinson's disease and related movement disorders (e.g. Huntington's Disease). A₂ₐ antagonists appear to demonstrate a reduced side-effect liability (e.g. no dyskinesia) compared to current dopaminergic therapies resulting in an improved therapeutic index. A₂ₐ antagonists may also have antidepressant properties and stimulate cognitive functions. Some xanthine-related compounds have been found to be A₁ receptor selective antagonists, and xanthine and non-xanthine compounds have been found to have high A₂ₐ affinity with varying degrees of A₂ₐ vs. A₁ selectivity. Adenosine A₂ₐ receptor antagonists have been disclosed previously, for example in WO 95/01356 and U.S. Pat. No. 6,630,475.

### SUMMARY OF THE INVENTION

This invention relates to a method for the treatment or prevention of Extra-Pyramidal Syndrome (e.g., dystonia, akathisia, pseudoparkinsonism and tardive dyskinesia) comprising administering a therapeutically effective amount of an adenosine A₂ₐ receptor antagonist to a patient in need thereof. In particular, this method is for the treatment or prevention of EPS in patients treated with an antipsychotic agent that has the side effect of inducing EPS. The adenosine A₂ₐ receptor antagonist can be administered after the symptoms of EPS have manifested, or an adenosine A₂ₐ receptor antagonist can be administered at the onset of administering an antipsychotic agent in order to prevent EPS from occurring. The invention, therefore, also includes a method of treating or preventing EPS induced by an antipsychotic agent comprising administering a combination of an antipsychotic agent and an adenosine A₂ₐ antagonist to a patient in need thereof. More particularly, the invention relates to the method of using of certain adenosine A₂ₐ antagonists for the monotherapy or the combined therapy.

The invention also relates to the treatment of primary (idiopathic) dystonia, and to the treatment or prevention of dystonia in patients who exhibit dystonia as a result of treatment with a tricyclic antidepressant, lithium or an anticonvulsant, or who have used cocaine, comprising administering a therapeutically effective amount of an adenosine A₂ₐ receptor antagonist to a patient in need thereof. When dystonia is caused by treatment with a tricyclic antidepressant, lithium or an anticonvulsant, the adenosine A₂ₐ receptor antagonist can be administered after the symptoms of dystonia have manifested, or an adenosine A₂ₐ receptor antagonist can be administered at the onset of administering a tricyclic antidepressant, lithium or an anticonvulsant in order to prevent dystonia from occurring. The invention, therefore, also includes a method of treating or preventing dystonia induced by a tricyclic antidepressant, lithium or an anticonvulsant comprising administering a combination of an adenosine A₂ₐ antagonist and a tricyclic antidepressant, lithium or an anticonvulsant to a patient in need thereof.

The invention also relates to the treatment of RLS or PLMS, comprising administering to a patient in need thereof a therapeutically effective amount of an adenosine A₂ₐ receptor antagonist. The invention also comprises a method of treating RLS or PLMS comprising administering a combination of an adenosine A₂ₐ antagonist with another agent useful in treating RLS or PLMS, such as levodopa/carbidopa, levodopa/benserazide, a dopamine agonist, a benzodiazepine, an opioid, an anticonvulsant or iron, to a patient in need thereof.

In another aspect, this invention relates to a kit comprising, in separate containers in a single package, pharmaceutical compositions for use in combination to treat or prevent EPS caused by treatment with antipsychotic agent, wherein one container comprises a pharmaceutical composition comprising an effective amount of an adenosine A₂ₐ receptor antagonist in a pharmaceutically acceptable carrier, and wherein a separate container comprises a pharmaceutical composition comprising an effective amount of an antipsychotic agent.

In another aspect, this invention relates to a kit comprising, in separate containers in a single package, pharmaceutical compositions for use in combination to treat or prevent dystonia caused by treatment with a tricyclic antidepressant, lithium or an anticonvulsant, wherein one container comprises a pharmaceutical composition comprising an effective amount of an adenosine A₂ₐ receptor antagonist in a pharmaceutically acceptable carrier, and wherein a separate container comprises a pharmaceutical composition comprising an effective amount of a tricyclic antidepressant, lithium or an anticonvulsant.

In another aspect, this invention relates to a kit comprising, in separate containers in a single package, pharmaceutical compositions for use in combination to treat RLS or PLMS, wherein one container comprises a pharmaceutical composition comprising an effective amount of an adenosine A₂ₐ receptor antagonist in a pharmaceutically acceptable carrier, and wherein a separate container comprises a pharmaceutical composition comprising an effective amount of levodopa/carbidopa, levodopa/benserazide, a dopamine agonist, a benzodiazepine, an opioid, an anticonvulsant or iron.

In yet another aspect, this invention relates to a fixed-dose pharmaceutical composition for treating or preventing EPS consisting of a therapeutically effective amount of a combination of an adenosine A₂ₐ receptor antagonist and an antipsychotic agent and a pharmaceutically acceptable carrier. Furthermore, the invention relates to a fixed-dose pharmaceutical composition for treating or preventing dystonia caused by treatment with lithium or an anticonvulsant consisting of a therapeutically effective amount of a combination of an adenosine A₂ₐ receptor antagonist and lithium or an anticonvulsant and a pharmaceutically acceptable carrier. The invention also relates to a fixed-dose pharmaceutical composition for treating RLS or PLMS consisting of a therapeutically effective amount of a combination of an adenosine A₂ₐ receptor antagonist and an opioid, an anticonvulsant or iron and a pharmaceutically acceptable carrier.

The invention also relates to the use of an adenosine A₂ₐ receptor antagonist for the preparation of a medicament for treating or preventing EPS, dystonia, RLS or PLMS, alone or in combination with the other agents discussed above.

### DETAILED DESCRIPTION OF THE DRAWINGS

A more complete understanding of the invention may be obtained by reading the following description in conjunction with the appended figures relating to haloperidol-induced EPS in Cebus apella monkeys.

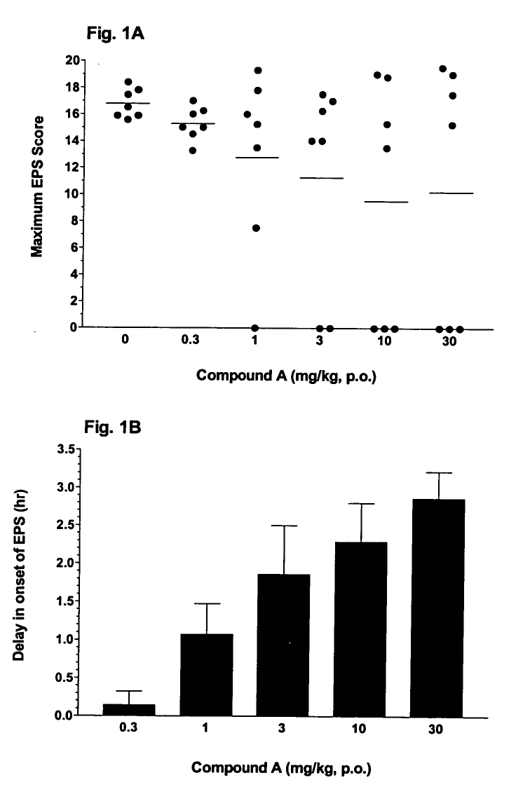

FIG. 1A illustrates the effect of Compound A (1-30 mg/kg, p.o.) on maximum EPS score.

FIG. 1B represents the mean delay in onset of EPS for each treatment group using Compound A compared to a vehicle control group.

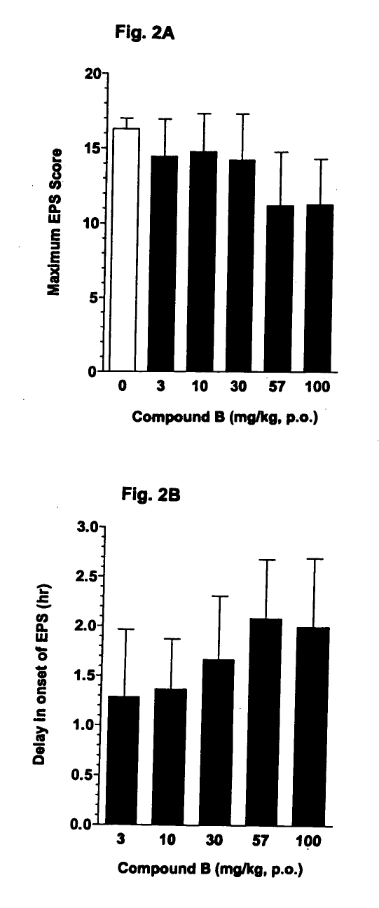

FIG. 2A illustrates the effect of Compound B (3-100 mg/kg, p.o.) on maximum EPS score.

FIG. 2B represents the mean delay in onset of EPS for each treatment group using Compound B compared to a vehicle control group.

### DETAILED DESCRIPTION OF THE INVENTION

Any adenosine A₂ₐ receptor antagonist is contemplated for use in the method of this invention. Suitable adenosine A₂ₐ receptor antagonists useful in the method of the invention can be identified by the binding assay described below. Specific examples of suitable adenosine A₂ₐ antagonists include the compounds disclosed in several patents and patent applications, e.g. WO 95/01356; U.S. Pat. Nos. 5,565,460; 6,630,475 B2; 5,935,964; 6,653,315; 6,916,811; US 2003/0212080; U.S. Pat. Nos. 6,875,772; 6,787,541 B1. Specifically, these patents and applications disclose the following compounds.

U.S. Pat. No. 6,630,475 B2 discloses compounds having the structural formula I 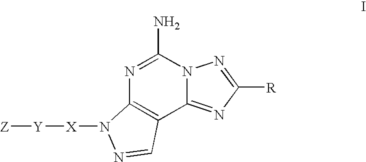
or a pharmaceutically acceptable salt thereof, wherein

R is R¹-furanyl, R¹-thienyl, R¹-pyridyl, R¹-pyridyl N-oxide, R¹-oxazolyl, R¹⁰-phenyl, R¹-pyrrolyl or C₄-C₆ cycloalkenyl;

X is C₂-C₆ alkylene or —C(O)CH₂—;

Y is —N(R²)CH₂CH₂N(R³)—, —OCH₂CH₂N(R²)—, —O—, —S—, —CH₂S—, —(CH₂)₂—NH—, or 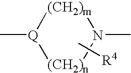
and

Z is R⁵-phenyl, R⁵-phenyl(C₁-C₆)alkyl, R⁵-heteroaryl, diphenylmethyl, R⁶—C(O)—, R⁶—SO₂—, R⁶—OC(O)—, R⁷—N(R⁸)—C(O)—, R⁷—N(R⁸)—C(S)—, 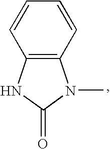
phenyl-CH(OH)—, or phenyl-C(═NOR²)—; or when Q is 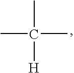
Z is also phenylamino or pyridylamino; or

Z and Y together are

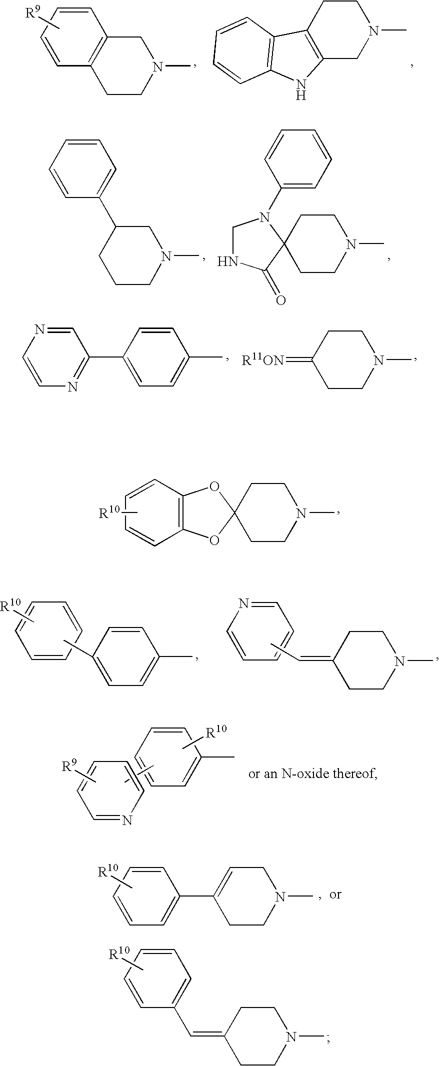

R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, and C₁-C₆ alkylsulfonyl;

R² and R³ are independently selected from the group consisting of hydrogen and C₁-C₆ alkyl;

m and n are independently 2-3;

Q is

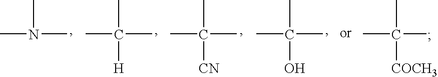

R⁴ is 1-2 substituents independently selected from the group consisting of hydrogen and C₁-C₆alkyl, or two R⁴ substituents on the same carbon can form ═O;

R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy, 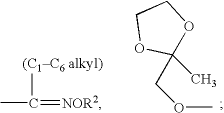
or adjacent R⁵ substituents together are —O—CH₂—O—, —O—CH₂CH₂—O—, —O—CF₂—O— or —O—CF₂CF₂—O— and form a ring with the carbon atoms to which they are attached;

R⁶ is (C₁-C₆)alkyl, R⁵-phenyl, R⁵-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or

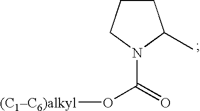

R⁷ is (C₁-C₆)alkyl, R⁵-phenyl or R⁵-phenyl(C₁-C₆)alkyl;

R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;

R⁹ is 1-2 groups independently selected from hydrogen, C₁-C₆ alkyl, hydroxy, C₁-C₆ alkoxy, halogen, —CF₃ and (C₁-C₆)alkoxy(C₁-C₆)alkoxy;

R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃ and —S(O)₀₋₂(C₁-C₆)alkyl;

R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;

R¹² is H or C₁-C₆ alkyl; and

R¹³ is (C₁-C₆)alkyl-C(O)— or (C₁-C₆)alkyl-SO₂—.

Preferred compounds of formula I are those wherein R is R¹-furanyl, R¹-thienyl, R¹-pyrrolyl or R¹⁰-phenyl, more preferably R¹-furanyl. R¹ is preferably hydrogen or halogen. Another group of preferred compounds is that wherein X is alkylene, preferably ethylene. Y is preferably 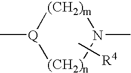
wherein Q is 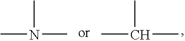
with Q preferably being nitrogen. Preferably, m and n are each 2, and R⁴ is H. A preferred definition for Z is R⁵-phenyl, R⁵-heteroaryl, R⁶—C(O)— or R⁶—SO₂—. R⁵ is preferably H, halogen, alkyl, alkoxy, hydroxyalkoxy or alkoxyalkoxy. R⁶ is preferably R⁵-phenyl.

Preferred specific compounds of formula I are those of the formula IA

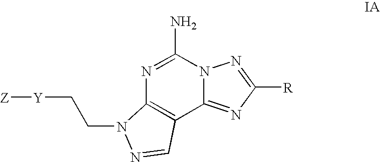

wherein R and Z-Y are as defined in the following table:

| Z—Y— | R |
|---|---|
| 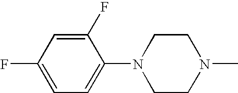 | 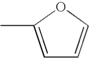 |
| 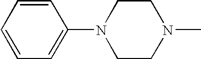 | 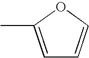 |
| 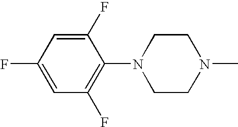 | 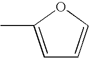 |
| 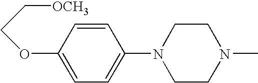 | 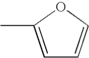 |
| 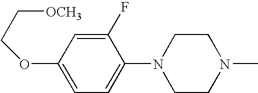 | 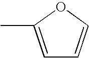 |
| 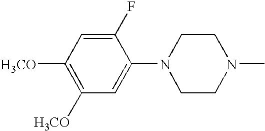 | 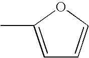 |
| 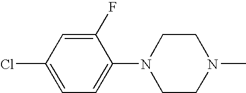 | 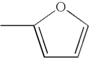 |
| 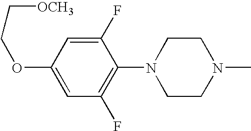 | 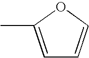 |
| 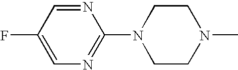 | 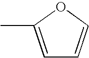 |
| 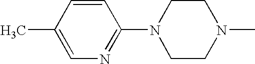 | 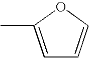 |
| 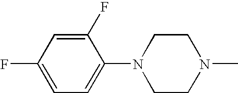 | 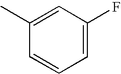 |
| 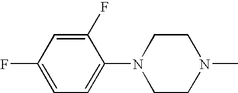 | 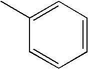 |

Other useful adenosine A₂ₐ receptor antagonists include those disclosed in WO 95/01356 as compounds having the structural formula II 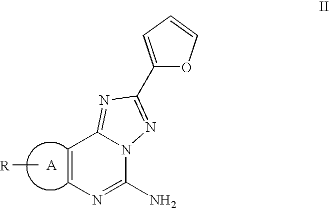
wherein:

A is pyrazole, imidazole or a triazole ring;

R is hydrogen; C₁-C₈ alkyl; C₃-C₇ alkenyl; C₃-C₇ alkynyl; C₃-C₇ cycloalkyl; C₁-C₅ alkyl substituted with one or more halogen atoms, hydroxy groups, C₁-C₄ alkoxy, C₃-C₇ cycloalkyl, groups of formula —NR₁R₂, —CONR₁R₂; aryl optionally substituted with halogen atoms, C₁-C₄ alkoxy groups, C₁-C₄ alkyl, nitro, amino, cyano, C₁-C₄ haloalkyl, C₁-C₄ haloalkoxy, carboxy, carboxyamido; C₇-C₁₀ aralkyl in which the aryl moiety can be substituted with one or more of the substituents indicated above for the aryl group; a group of formula —(CH₂)ₘ-Het, wherein Het is a 5-6 membered aromatic or non aromatic heterocyclic ring containing one or more heteroatoms selected from N, O, S and m is an integer from 1 to 5;

R₁, R₂ which are the same or different, are hydrogen, C₁-C₅ alkyl, C₇-C₁₀ aralkyl, phenyl, or taken together with the nitrogen they are linked to, form an azetidine ring or a 5-6 membered heterocyclic ring containing one or more heteroatoms such as N, O, S and n is an integer from 2 to 5.

Preferably, compounds of formula II are those wherein R is hydrogen, C₁-C₈ alkyl, aryl or C₇-C₁₀ aralkyl optionally substituted, preferably with halogen atoms.

U.S. Pat. No. 5,935,964 discloses useful adenosine A₂ₐ receptor antagonist compounds having the structural formula III 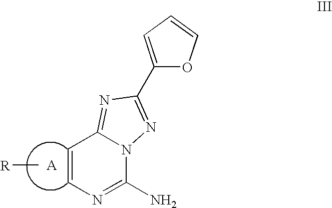
wherein A is pyrazole, imidazole or triazole ring;

R is

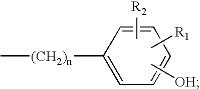

R₁ and R₂, which are the same or different, are H, OH, halogen, C₁-C₄ alkoxy, C₁-C₄ alkyl, nitro, amino, cyano, C₁-C₄ haloalkyl, C₁-C₄ haloalkoxy, carboxy or carboxamido; or the OH group, together with one of R₁ or R₂, or R₁ and R₂, can form a methylenedioxy group —O—CH₂—O—; and

n is an integer from 0-4.

Preferred compounds of formula III are those wherein A is pyrazolo[4,3-e] or 1,2,3-triazolo[5,4-e].

U.S. Pat. No. 5,565,460 discloses useful adenosine A₂ₐ receptor antagonist compounds having the structural formulas IVA and IVB, wherein formula IVA is 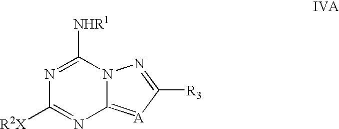
wherein R¹ represents hydrogen, substituted or unsubstituted lower alkyl, or substituted or unsubstituted lower alkanoyl;

R² represents hydrogen, substituted or unsubstituted lower alkyl, substituted or unsubstituted lower alkenyl, substituted or unubstituted cycloalkyl, substituted or unsubstituted aryl, substituted or unsubstituted aralkyl, or a substituted or unsubstituted heterocyclic group;

R³ represents a substituted or unsubstituted heterocyclic group;

X represents a single bond, O, S, S(O), S(O)₂, or NR⁴ (in which R⁴ represents hydrogen, or substituted or unsubstituted lower alkyl; or R² and NR⁴ are combined to form a substituted or unsubstituted 4 to 6-membered saturated heterocyclic group): and

A represents N or CR⁵ (in which R⁵ represents hydrogen, or a substituted or unsubstituted lower alkyl); and
wherein formula IVB is 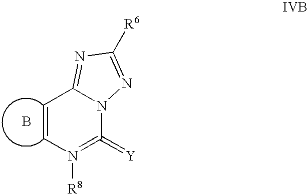
wherein R⁶ represents substituted or unsubstituted aryl, or a substituted or unsubstituted heterocyclic group;

Y represents O, S, or NR⁷ (in which R⁷ represents substituted or unsubstituted lower alkyl, substituted or unubstituted cycloalkyl, or substituted or unsubstituted aryl);

R⁸ represents hydrogen, substituted or unsubstituted lower alkyl, substituted or unsubstituted lower alkenyl, substituted or unsubstituted lower alkynyl, substituted or unubstituted cycloalkyl, substituted or unsubstituted aryl, substituted or unsubstituted aralkyl, or a substituted or unsubstituted heterocyclic group; and

B and the adjacent two carbon atoms are combined to form a substituted or unsubstituted, partially saturated or unsaturated, monocyclic or bicyclic, carbocyclic or heterocyclic group.

U.S. Pat. No. 6,653,315 discloses useful adenosine A₂ₐ receptor antagonist compounds having the structural formula V 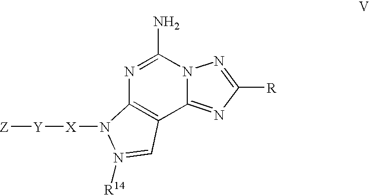
or a pharmaceutically acceptable salt thereof, wherein

R is R¹-heteroaryl, R¹⁰-phenyl, C₄-C₆ cycloalkenyl, —C(═CH₂)CH₃, —C≡C—CH₃, —C≡C—CH₂—OR², —CH═C(CH₃)₂,

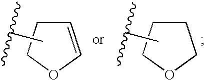

X is C₁-C₆ alkylene, —C(O)CH₂— or —C(O)N(R²)CH₂—;

Y is —N(R²)CH₂CH₂N(R³)—, —OCH₂CH₂N(R²)—, —O—, —S—, —CH₂S—, —(CH₂)₂₋₃—N(R²)—, R⁵-divalent heteroaryl, 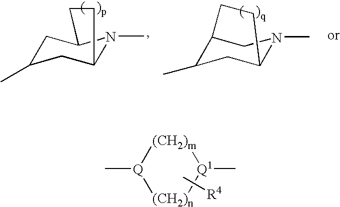
and

Z is R⁵-phenyl, R⁵-phenyl(C₁-C₆)alkyl, R⁵-heteroaryl, R⁵-bicyclic heteroaryl, R⁵-benzofused heteroaryl, diphenylmethyl or R⁶—C(O)—;
or when Y is 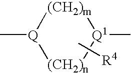
Z is also R⁶—SO₂—, R⁷—N(R⁸)—C(O)—, R⁷—N(R⁸)—C(S)— or R⁶OC(O)—;
or when Q is 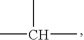
Z is also phenylamino or pyridylamino;
or Z and Y together are 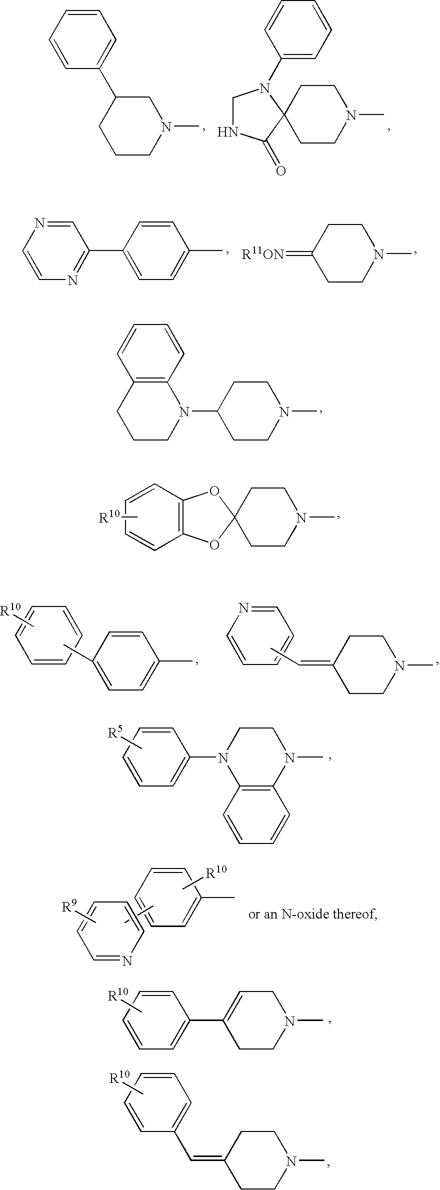
or Y and Z together form a piperidinyl or pyrrolidinyl ring fused to a monocyclic or bicyclic aryl or a monocyclic or bicyclic heteroaryl ring wherein X is attached to the N atom of the piperidinyl or pyrrolidinyl ring;

R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, C₁-C₆ alkylsulfonyl, —COOR⁷ or —C(O)NR²R³;

R² and R³ are independently selected from the group consisting of hydrogen and C₁-C₆ alkyl;

m and n are independently 2-3;

p and q are independently 0-2;

Q and Q¹ are independently selected from the group consisting of 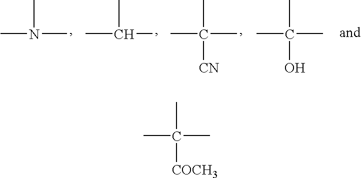
provided that at least one of Q and Q¹ is

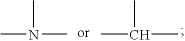

R⁴ is 1-2 substituents independently selected from the group consisting of hydrogen, C₁-C₆alkyl, R¹-aryl and R¹-heteroaryl, or two R⁴ substituents on the same carbon can form ═O;

R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO₂—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy, 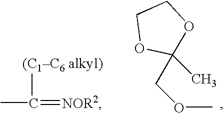
(R²O)₂—P(O)—CH₂—O— and (R²O)₂—P(O)—; or adjacent R⁵ substituents together are —O—CH₂—O—, —O—CH₂CH₂—O—, —O—CF₂—O— or —O—CF₂CF₂—O— and form a ring with the carbon atoms to which they are attached;

R⁶ is (C₁-C₆)alkyl, R⁵-phenyl, R⁵-phenyl(C₁-C₆)alkyl, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, or

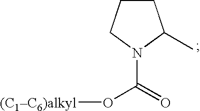

R⁷ is (C₁-C₆)alkyl, R⁵-phenyl or R⁵-phenyl(C₁-C₆)alkyl;

R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;

R⁹ is 1-2 substituents independently selected from the group consisting of hydrogen, C₁-C₆ alkyl, hydroxy, C₁-C₆ alkoxy, halogen, —CF₃ and (C₁-C₆)alkoxy-(C₁-C₆)alkoxy;

R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃, —S(O)₀₋₂(C₁-C₆)alkyl and —CH₂—SO₂-phenyl;

R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;

R¹² is H or C₁-C₆ alkyl;

R¹³ is H, (C₁-C₆)alkyl-C(O)— or (C₁-C₆)alkyl-SO₂—;

R¹⁴ is H, halogen, C₁-C₆ alkyl, hydroxy(C₁-C₆)alkyl, C₁-C₆ alkoxy(C₁-C₆)alkyl, thio(C₁-C₆)alkyl, (C₁-C₆)alkylthio(C₁-C₆)alkyl or NR²R³—(C₁-C₆)alkyl; and

R¹⁵ is H, halogen, C₁-C₆ alkyl or C₁-C₆ alkoxy.

Preferred compounds of formula V are those wherein R is R¹-furanyl, R¹-thienyl, R¹-pyrrolyl, R¹-pyridyl or R¹⁰-phenyl, more preferably R¹-furanyl or R¹⁰-phenyl. R¹ is preferably hydrogen or halogen. R¹⁰ is preferably hydrogen, halogen, alkyl or —CF₃. Another group of preferred compounds is that wherein X is alkylene, preferably ethylene. Y is preferably 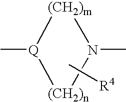
wherein Q is 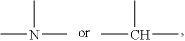
with Q preferably being nitrogen. Preferably, m and n are each 2, and R⁴ is H. A preferred definition for Z is R⁵-phenyl or R⁵-heteroaryl. R⁵ is preferably H, halogen, alkyl, alkoxy, hydroxyalkoxy or alkoxyalkoxy. R⁶ is preferably R⁵-phenyl.

Preferred specific compounds of formula V are those of the formula VA

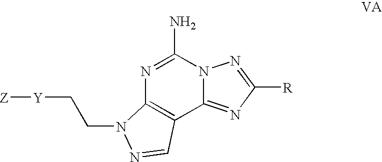

wherein R and Z-Y are as defined in the following table:

| Z—Y— | R |
|---|---|
| 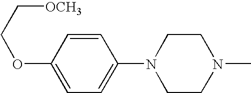 | 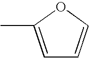 |
| 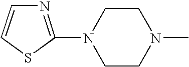 | 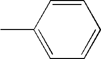 |
| 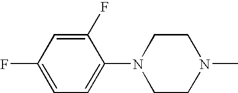 | 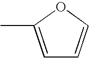 |
| 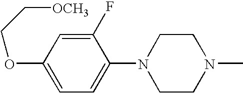 | 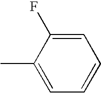 |
| 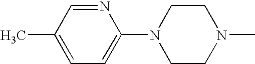 | 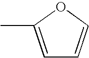 |
| 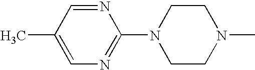 | 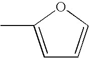 |
| 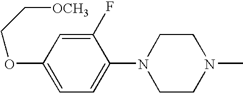 | 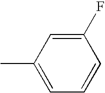 |
| 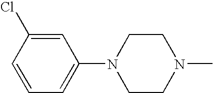 | 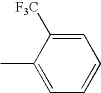 |
| 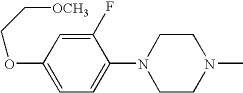 | 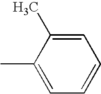 |
| 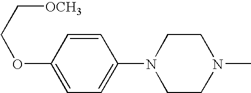 | 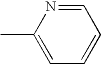 |

U.S. Pat. No. 6,916,811 discloses useful adenosine A₂ₐ receptor antagonist compounds having the structural formula VI 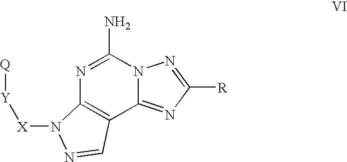
or a pharmaceutically acceptable salt or solvate of said compound, wherein:

R is selected from the group consisting of R¹-furanyl-, R¹-thienyl-, R¹-pyridyl-, R¹-oxazolyl-, R¹-pyrrolyl- and R²-aryl-;

X is —(CH₂)ₙ—;

Y is a piperidinyl, pyrrolidinyl or azepanyl group with an aryl or heteroaryl moiety fused to two adjacent carbon atoms on Y, wherein X is attached to the N atom of the piperidinyl, pyrrolidinyl or azepanyl group;

Q is 1-4 substituents, which can be the same or different, and are independently selected from the group consisting of hydrogen, cycloalkyl, cycloheteroalkyl, amino, aryl, aralkyl, heteroaryl, alkyl, CF₃, CN, halogen, NO₂, alkoxy, alkoxyalkoxy, cycloalkylalkoxy, acyloxy, alkylamino, acylamino, alkylsulfonamino, alkylaminosulfonyl, dialkylaminosulfonyl, NH₂SO₂—, and hydroxy;

n is 1 to 4;

R¹ is 1-3 substituents, which may be the same or different, and are independently selected from the group consisting of hydrogen, alkyl, CF₃, halogen and NO₂; and

R² is 1-3 substituents, which may be the same or different, and are independently selected from the group consisting of hydrogen, alkyl, CF₃, halogen, NO₂, alkoxy, acyloxy, alkylamino, acylamino, alkylsulfonamido, alkylaminosulfonyl, dialkylaminosulfonyl, aminosulfonyl, and hydroxyl.

In a preferred embodiment of compounds of formula VI, Y is

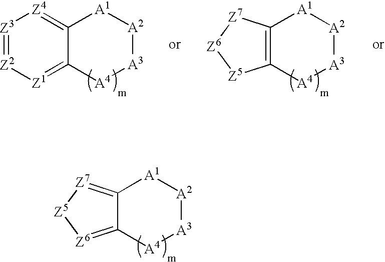

wherein A¹ is N—X, and A² and A³ each are CR⁴R⁵, or
A¹ and A³ each are CR⁴R⁵, and A² is N—X, or
A¹ and A² each are CR⁴R⁵, and A³ is N—X;

A⁴ is CR⁴R⁵;

Z¹, Z², Z³ and Z⁴, which can the same or different, are each independently selected from the group consisting of N and CR³, provided that 0-2 of Z¹, Z², Z³ or Z⁴ are N and the remainder are CR³;

Z⁵ is NR⁵, O, S or CR⁴R⁵;

Z⁶ is N or CR³;

Z⁷ is N or CR³;

m is an integer from 0 to 2;

R³ is selected from the group consisting of hydrogen, cycloalkyl, amino, aryl, heteroaryl, C₁-C₆-alkyl, CF₃, CN, halogen, NO₂, C₁-C₆-alkoxy, C₁-C₆-acyloxy, C₁-C₆-alkylamino, C₁-C₆-acylamino, C₁-C₆-alkylsulfonamino, C₁-C₆-alkylaminosulfonyl, C₁-C₆-dialkylaminosulfonyl, NH₂—SO₂—, and hydroxy;

R⁴ is selected from the group consisting of hydrogen, hydroxyalkyl, aryl, aralkyl, C₁-C₆-alkyl, C₁-C₆-alkoxy, CF₃, CN, halogen, hydroxy, and NO₂; and

R⁵ is hydrogen or C₁-C₆ alkyl.

Preferred specific examples of compounds of formula VI include compounds of the formula:

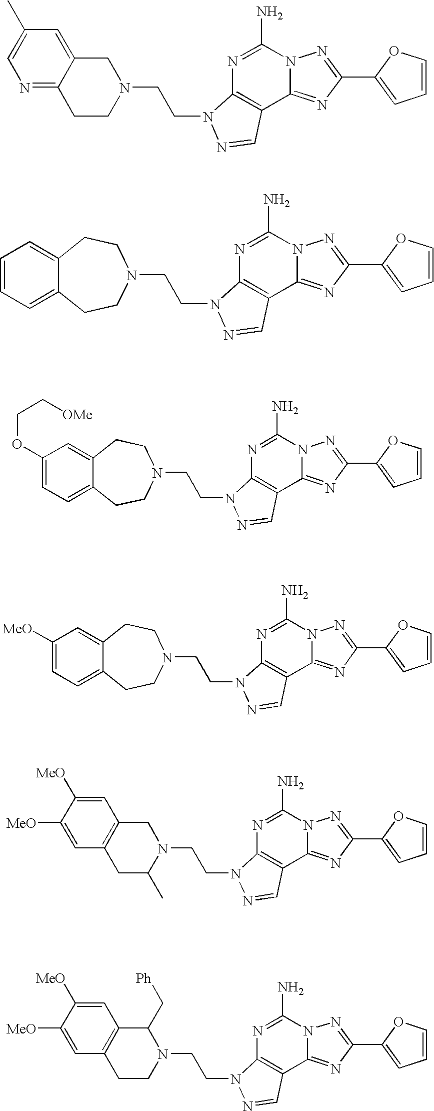

US2003/0212080 discloses useful adenosine A₂ₐ receptor antagonist compounds having the structural formula VII 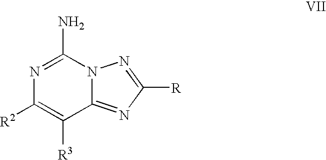
or a pharmaceutically acceptable salt or solvate thereof; wherein:

R is selected from the group consisting of R⁴-heteroaryl, R⁵-phenyl, (C₄-C₆)cycloalkenyl, —C(═CH₂)CH₃, —C≡C—CH₃, 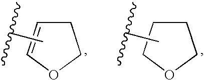
—CH═C(CH₃)₂, 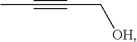
and —CH═CH—CH₃;

R² is selected from the group consisting of —W—X, NR¹⁹(CH₂)ₘ—W—X, and —NR¹⁹CH(CH₃)—W—X, or

R² is selected from the group consisting of alkyl, alkenyl and —NR¹⁸R¹⁹, wherein said alkyl, alkenyl or —NR¹⁸R¹⁹ is optionally substituted by —W—X;

R³ is selected from the group consisting of H, halo, alkyl, trifluoromethyl, alkoxy, alkoxyalkyl, hydroxyalkyl, alkylamino, alkylaminoalkyl, dialkylamino, dialkylaminoalkyl, aminoalkyl, aryl, heteroaryl, and CN;

R⁴ is 1 to 3 substituents, which can be the same or different, and are independently selected from the group consisting of hydrogen, (C₁-C₆)-alkyl, —CF₃, halogen, —NO₂, —NR¹⁵R¹⁶, (C₁-C₆)alkoxy, (C₁-C₆)alkylthio, (C₁-C₆)alkylsulfinyl, (C₁-C₆)alkylsulfonyl, —COOR¹⁷ and —C(O)NR⁶R⁷;

R⁵ is 1 to 5 substituents, which can be the same or different, and are independently selected from the group consisting of hydrogen, halogen, (C₁-C₆)alkyl, hydroxy, (C₁-C₆)alkoxy, —CN, —NH₂, (C₁-C₆)alkylamino, di-((C₁-C₆)alkyl)amino, —CF₃, —OCF₃, —S(O)₀₋₂(C₁-C₆)alkyl and —CH₂—SO₂-phenyl;

R⁶ and R⁷, which can be the same or different, are each independently selected from the group consisting of hydrogen and (C₁-C₆)alkyl;

R⁸ is 1 to 5 substituents, which can be the same or different, and are independently selected from the group consisting of hydrogen, halogen, (C₁-C₆)alkyl, hydroxy, C₁-C₆ alkoxy, —CN, amino, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO₂—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy, 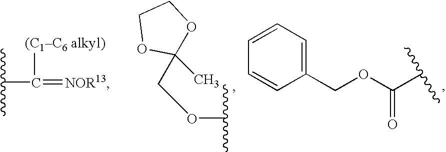
—O—CH₂—P(O)(OR⁶)₂,- and —P(O)(OR⁶)₂; or

adjacent R⁸ substituents together are —O—CH₂—O—, —O—CH₂CH₂—O—, —O—CF₂—O—or

—O—CF₂CF₂—O— and form a ring with the carbon atoms to which they are attached;

R⁹ is selected from the group consisting of (C₁-C₆)alkyl, R⁸-aryl-, R⁸-aryl(C₁-C₆)alkyl-, thienyl, pyridyl, (C₃-C₆)-cycloalkyl, (C₁-C₆)alkyl-OC(O)—NH—(C₁-C₆)alkyl-, di-((C₁-C₆)alkyl)aminomethyl, cycloheteroalkyl(C₁-C₆)alkyl, aryloxy(C₁-C₆)alkyl, alkoxy(C₁-C₆)alkyl and

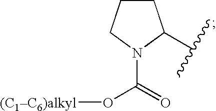

R¹⁰ is 1-2 substituents, which can be the same or different, and are independently selected from the group consisting of hydrogen, (C₁-C₆)alkyl, R⁵-aryl and R⁴-heteroaryl, or two R¹⁰ substituents on the same carbon can form ═O;

R¹¹ is hydrogen or (C₁-C₆)alkyl; —C(O)alkyl, or R¹⁷ and R¹¹ taken together are —(CH₂)ₚ-A-(CH₂)_{q}, wherein p and q are each independently 2 or 3 and A is selected from the group consisting of a bond, —CH₂—, —S— and —O—, and form a ring with the nitrogen to which they are attached;

R¹² is 1-2 substituents, which can be the same or different, and are independently selected from the group consisting of hydrogen, (C₁-C₆)alkyl, hydroxy, (C₁-C₆)alkoxy, halogen, and —CF₃;

R¹³ is selected from the group consisting of H, (C₁-C₆)alkyl, phenyl, benzyl, (C₂-C₆)alkenyl, (C₁-C₆)alkoxy(C₁-C₆)alkyl, di-((C₁-C₆)alkyl)amino(C₁-C₆)alkyl, pyrrolidinyl(C₁-C₆)alkyl and piperidino(C₁-C₆)alkyl;

R¹⁴ is selected from the group consisting of H, halogen, (C₁-C₆)alkyl or (C₁-C₆)alkoxy;

R¹⁵ is selected from the group consisting of H and (C₁-C₆)alkyl;

R¹⁶ is selected from the group consisting of H, (C₁-C₆)alkyl-C(O)— and (C₁-C₆)alkyl-SO₂—;

R¹⁷ is selected from the group consisting of (C₁-C₆)alkyl, (C₁-C₆)hydroxyalkyl, (C₃-C₆)cycloalkyl, (C₁-C₆)alkoxy(C₁-C₆)alkoxy, (C₁-C₆)alkoxy, (C₁-C₆)alkoxy(C₁-C₆)alkyl, allyl, propargyl, R⁸-heteroaryl-, R⁸-aryl- and R⁸-aryl(C₁-C₆)alkyl-;

R¹⁸ is selected from the group consisting of a bond, —CH₂—, —CH(OH)—, —CH(CH₃)—, —C(CH₃)ₙ—, —(CH₂)ₙ—, and —O(CH₂)ₙ—, R¹⁹ is selected from the group consisting of H, (C₁-C₆)alkyl, (C₁-C₆)alkyl(C₁-C₆)cycloalkyl, (C₁-C₆)cycloalkyl(C₁-C₆)alkyl and (C₁-C₆)alkoxy(C₁-C₆)alkyl;

Q and Q¹ can be the same or different and are each independently selected from the group consisting of

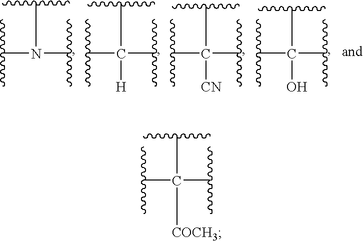

m and n are each independently 1-3;

p and q are each independently 0-2;

s is 0-4;

W is aryl or heteroaryl having 1-3 heteroatoms, which can be the same or different, and are independently selected from the group consisting of N, O and S, and wherein said aryl or heteroaryl is optionally substituted with 1-3 substituents, which can be the same or different, and are independently selected from the group consisting of alkyl, aryl, alkylcycloalkyl, halo, hydroxy, hydroxyalkyl, alkoxy, alkylalkoxy, alkoxyalkoxy, —NR⁶R⁷, (C₂-C₆)alkene, and —CN, or

X is selected from the group consisting of H, NH₂, —N(R⁶)(CH₂)ₛ-aryl, —N(R⁶)(CH₂)ₛ-heteroaryl, —N(R⁶)(CH₂)ₘ₊₁—OH, and —N(CH₃)₂, or

X is —R¹⁸—Y-Z;

Y is selected from the group consisting of —N(R⁶)CH₂CH₂N(R⁷)—, —N(R⁶)(CH₂)ₙaryl, —OCH₂CH₂N(R⁶)—, —O—, —S—, —CH₂S—, —(CH₂)₂₋₃—N(R⁶)—, R⁸-divalent heteroaryl,

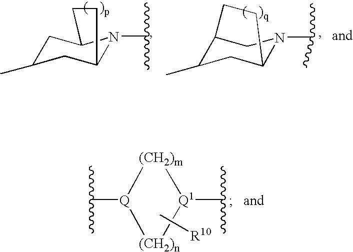

Z is selected from the group consisting of H, alkyl, alkoxyalkyl, R⁸-aryl-, R⁸-aryl(C₁-C₆)alkyl-, R⁸-heteroaryl-, R⁸-bicyclicalkyl-, aminoalkyl, alkylamino, NH₂, —N—(R⁶)(CH₂)ₛ-aryl, —N(R⁶)(CH₂)ₛ-heteroaryl, —N(R₆)C(O)OR¹⁷, alkylcycloheteroalkyl, cycloheteroalkyl, cycloheteroalkylalkyl, alkoxycycloheteroalkyl, heteroaryl; R⁸-benzofused heteroaryl-, diphenylmethyl and R⁹—C(O)—; or
when Y is 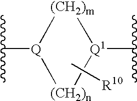
Z can also be —OH, R⁹—SO₂—, R¹⁷—N(R¹¹)(CH₂)ₛ—C(O)—, R¹⁷—OC(O)—, R¹⁷—O(CH₂)ₙC(O)—, benzofused heteroaryl(CH₂)ₙC(O)—, benzofused heteroaryl(CH₂)ₙ— or R¹⁷—N(R¹¹)—C(S)—; or

when Q is 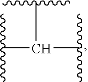
Z can also be R¹⁷R¹¹N—, phenylamino or pyridylamino; or

Z and Y taken together are selected from the group consisting of

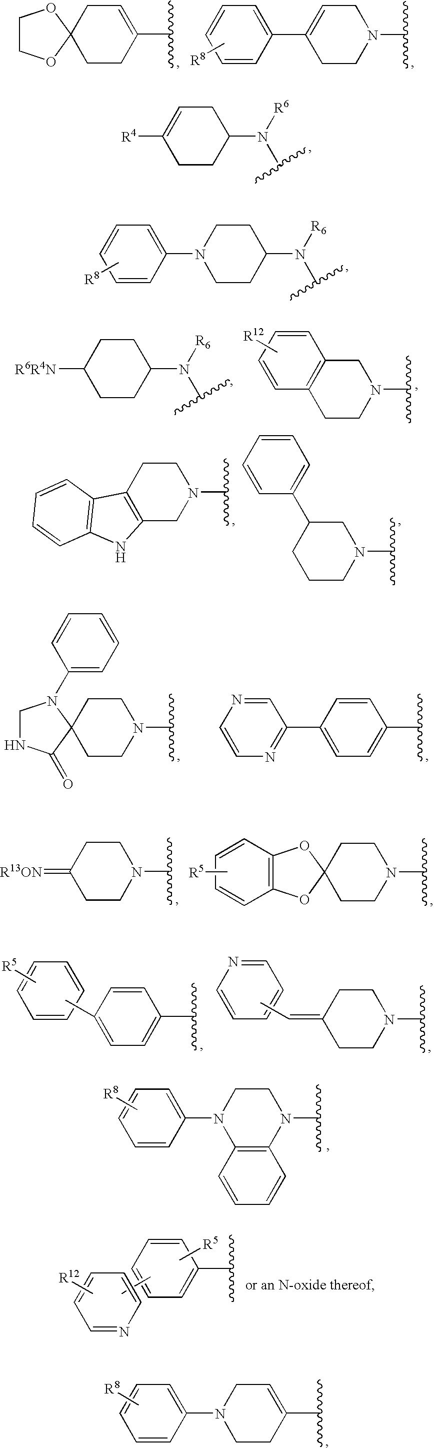

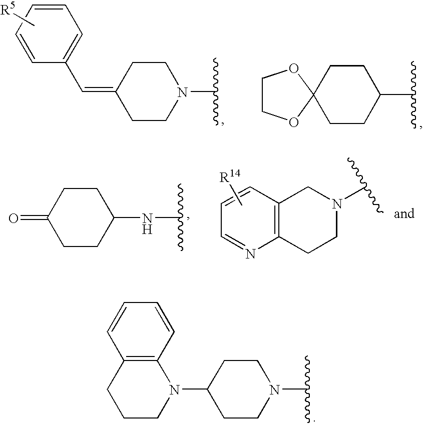

Preferred compounds of formula VII are those having the following structures:

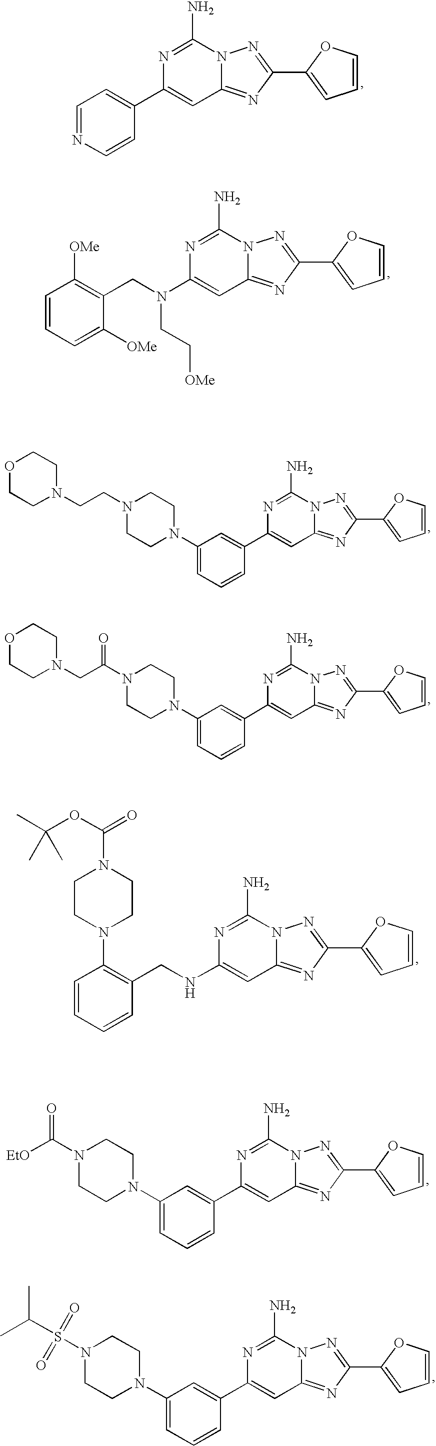

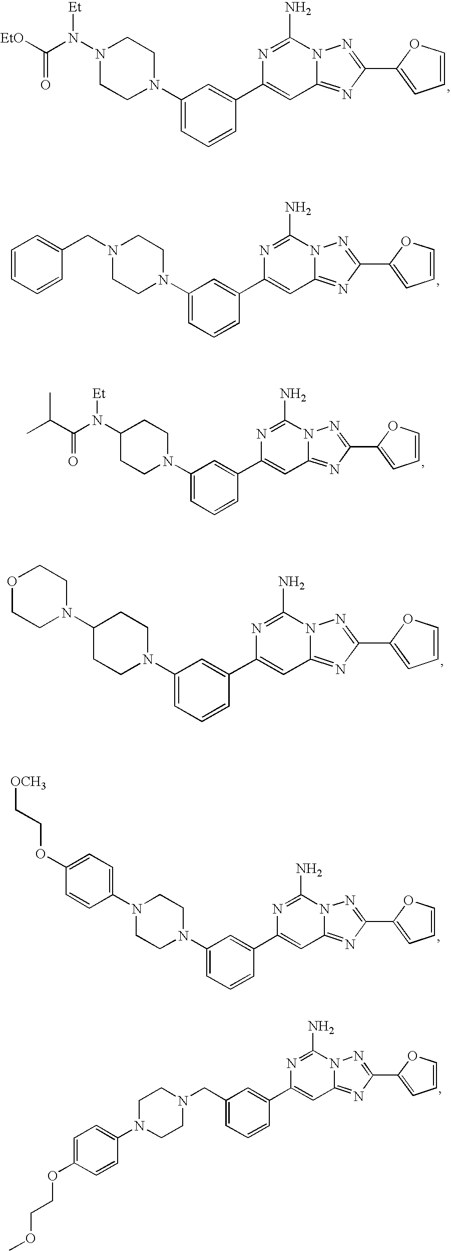

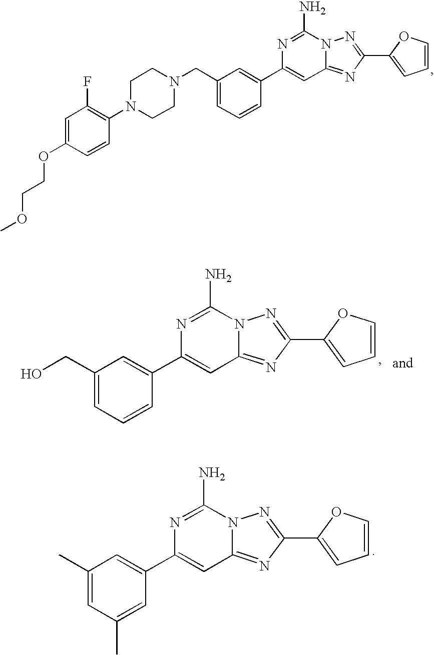

U.S. Pat. No. 6,875,772 discloses useful adenosine A₂ₐ receptor antagonist compounds having the structural formula VIII 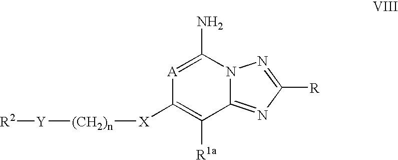
or a pharmaceutically acceptable salt thereof, wherein:

A is C(R¹) or N;

R¹ and R^{1a} are independently selected from the group consisting of H, (C₁-C₆)alkyl, halo, CN and —CF₃;

Y is —O—, —S—, —SO—, —SO₂—, R⁵-heteroaryldiyl, R⁵-arylene or

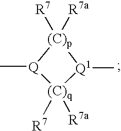

p and q are independently 2-3;

Q and Q¹ are independently selected from the group consisting of 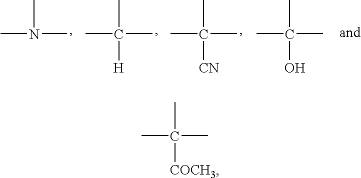
provided that at least one of Q and Q¹ is

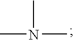

R is R⁵⁻aryl, R⁵⁻heteroaryl, R⁶—(C₂-C₆)alkenyl or R⁶—(C₂-C₆)alkynyl;

R² is R⁵⁻aryl, R⁵⁻heteroaryl, R⁵⁻aryl(C₁-C₆)alkyl or R⁵⁻heteroaryl(C₁-C₆)alkyl; or R²—Y is

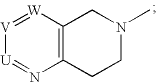

U, V, and W are independently selected from the group consisting of N and CR¹, provided that at least one of U, V and W is CR¹;

n is 1, 2 or 3; and
(a) A is C(R¹) and X is —C(R³)(R^{3a})—, —C(O)—, —O—, —S—, —SO—, —SO₂—, R⁴-arylene, R⁴-heteroaryldiyl, or —N(R⁹)—; or A is C(R¹), Y is a bond, and X is —C(R³)(R^{3a})—, —C(O)—, —O—, —S—, —SO—, —SO₂—, R⁴-arylene, —N(R⁹)— or R⁴-heteroaryldiyl, provided that when X is —N(R⁹)— or R⁴-heteroaryldiyl, R² is not phenyl or phenyl-(C₁-C₆)alkyl; or
(b) A is N, X is —N(R⁹)—, Y is R⁵-arylene and R² is

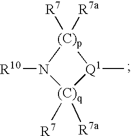

or n is 2 or 3; and
(c) A is N and X is —C(R³)(R^{3a})—, —C(O)—, —O—, —S—, —SO—, —SO₂—, —N(R⁹)—, R⁴-arylene or R⁴-heteroaryldiyl; or A is N, Y is a bond and X is —C(O)—, —N(R⁹)—, R⁴-arylene or R⁴-heteroaryldiyl; or A is N, Y is —N(R^{9a})—, —C(O)N(R^{9a})— or —O—(CH₂)₂—N(R^{9a})—, and X is —N(R⁹)—; or A is N, X is —N(R⁹)—, and Y and R² together are

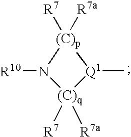

or n is 0; and
(d) A is N, Y is a bond, X is —N(R⁹)—, and R² is 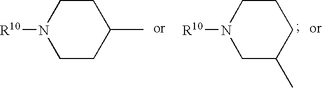
(e) A is N, X is —N(R⁹)— and Y and R² together are 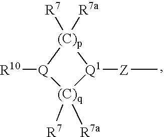
wherein Z is —C(O)—CH₂—, —C(O)—CH(C₁-C₆ alkyl)-, —CH₂—CH(C₁-C₆ alkyl)-, or —CH(C₁-C₆ alkyl)-CH₂—;

R³ and R^{3a} are independently selected from the group consisting of H, —OH, C₁-C₆ alkyl, hydroxy(C₁-C₆)alkyl, (C₁-C₆)alkoxy(C₁-C₆)alkyl, amino(C₁-C₆)alkyl, (C₁-C₆)alkylamino(C₁-C₆)alkyl and di(C₁-C₆)alkylamino(C₁-C₆)alkyl;

R⁴ is 1-3 substituents selected from the group consisting of H, (C₁-C₆)alkyl, —OH, (C₁-C₆)alkoxy, (C₁-C₆)alkoxy(C₁-C₆)alkoxy, halo, —CF₃, and —CN;

R⁵ is 1-3 substituents independently selected from the group consisting of H, (C₁-C₆)alkyl, —OH, (C₁-C₆)alkoxy, (C₁-C₆)alkoxy(C₁-C₆)alkyl, (C₁-C₆)alkoxy(C₁-C₆)alkoxy, halo, —CF₃, —CN, —NH₂, (C₁-C₆)alkylamino, di(C₁-C₆)alkylamino, amino(C₁-C₆)alkyl, (C₁-C₆)alkylamino(C₁-C₆)alkyl, di(C₁-C₆)alkylamino(C₁-C₆)alkyl, (C₁-C₆)alkanoylamino, (C₁-C₆)alkanesulfonylamino, (C₁-C₆)alkylthio, (C₁-C₆)alkylthio(C₁-C₆)alkyl, R⁶—(C₂-C₆)alkenyl, R⁶—(C₂-C₆)alkynyl, hydroxy(C₁-C₆)alkyl, (C₁-C₆)alkoxy-C(O)-amino, or heterocycloalkyl(C₁-C₆)alkyl;

R⁶ is 1 to 3 substituents independently selected from the group consisting of H, —OH, (C₁-C₆)alkoxy and halo;

R⁷ and R^{7a} are independently selected from the group consisting of H, (C₁-C₆)alkyl, (C₁-C₆)alkoxy(C₁-C₆)alkyl, R⁸-aryl and R⁸-heteroaryl, or an R⁷ and an R^{7a} substituent on the same carbon can form ═O;

R⁸ is 1 to 3 substituents independently selected from H, (C₁-C₆)alkyl, —OH, (C₁-C₆)alkoxy, (C₁-C₆)alkoxy(C₁-C₆)alkoxy, halo, —CF₃, and —CN;

R⁹ and R^{9a} are independently selected from the group consisting of H, (C₁-C₆)alkyl, hydroxy(C₂-C₆)alkyl, (C₁-C₆)alkoxy(C₂-C₆)alkyl, amino(C₂-C₆)alkyl, (C₁-C₆)alkylamino(C₂-C₆)alkyl, di(C₁-C₆)alkylamino(C₂-C₆)alkyl, halo-(C₃-C₆)alkenyl, CF₃—(C₁-C₆)alkyl, (C₃-C₆)alkenyl, (C₃-C₆)cycloalkyl and (C₃-C₆)cycloalkyl-(C₁-C₆)alkyl; and

R¹⁰ is H, —C(O)—O—(C₁-C₆)alkyl, R⁵-aryl, —C(O)—(C₁-C₆)alkyl, —C(O)—(R⁵-aryl) or R⁵-aryl-(C₁-C₆)alkyl.
Preferred compounds of formula VIII are those wherein A is N. R is preferably furyl. R^{1a} is preferably hydrogen. Another group of preferred compounds is that wherein X is —O—, —S—, —N(R⁹)— or R⁴-arylene, with compounds wherein X is —N(R⁹)— being more preferred. R⁹ is preferably C₁-C₆ alkyl. Preferred definitions for Y are a bond or piperazinyl. R² is preferably R⁵-aryl. When Y and/or R² is 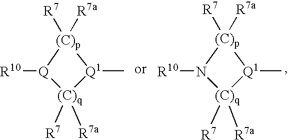
Q is preferably N, Q¹ is preferably N, p and q are each preferably 2, each R⁷ and R^{7a} is preferably hydrogen, and R¹⁰ is preferably —C(O)—O—(C₁-C₆)alkyl, —C(O)—(C₁-C₆)alkyl or —C(O)—(R⁵-aryl). R⁵ is preferably 1 or 2 substituents selected from the group consisting of H, (C₁-C₆)alkoxy, (C₁-C₆)alkoxy(C₁-C₆)-alkoxy, halo and —CF₃. R⁴ is preferably H, halo or (C₁-C₆)alkyl. R³ and R^{3a} are preferably independently selected from H and (C₁-C₆)alkyl. R^{9a} is preferably H or (C₁-C₆)alkyl. R⁶ is preferably hydrogen.

Preferred specific examples of compounds of formula VIII include compounds of the formula 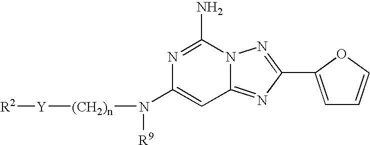
wherein R²—Y—(CH₂)ₙ—N(R⁹)— is as defined in the table:

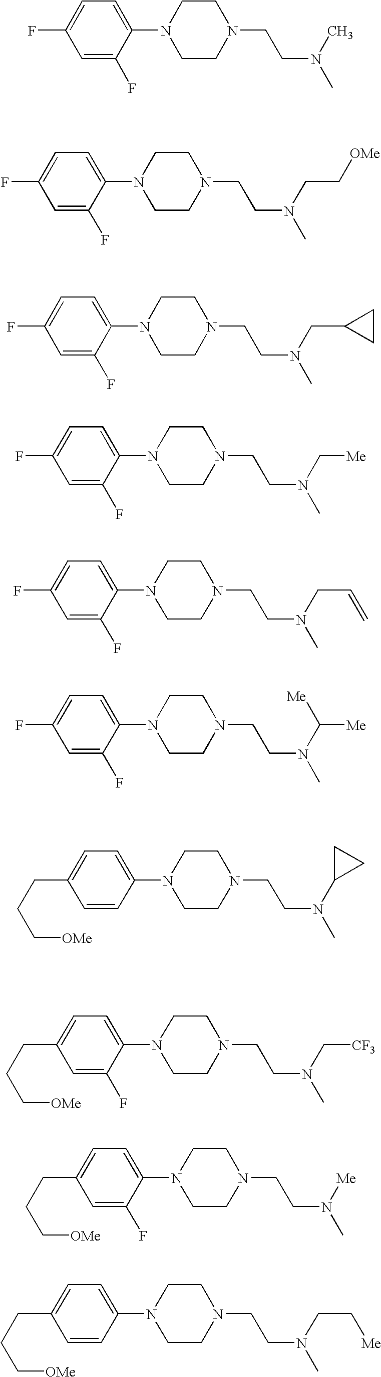

U.S. Pat. No. 6,787,541 discloses useful adenosine A₂ₐ receptor antagonist compounds having the structural formula IX 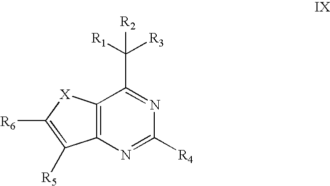
wherein

X is O or S;

R₁ and R₂ are independently selected from hydrogen, alkyl, aryl, hydroxy, alkoky, aryloxy, cyano, nitro, CO₂R₇, COR₇, OCOR₇, CONR₇R₈, CONR₇NR₈R₉, OCONR₇R₈, NR₇R₈, NR₇COR₈, NR₇CONR₈R₉, NR₇CO₂R₈, NR₇SO₂R₈, NR₇CONR₈NR₉R₁₀, NR₇NR₈CO₂R₉, NR₇NR₈CONR₉R₁₀, NR₇SO₂NR₈R₉, SO₂R₇, SOR₇, SR₇ and SO₂NR₇R₈, or R₁ and R₂ together form a carbonyl group (C═O), an oxime group (C═NOR₁₁), an imine group (C═NR₁₁) or a hydrazine group (C═NNR₁₁R₁₂), or R₁ and R₂ together form a 5, 6 or 7 membered carbocyclic or heterocyclic ring;

R₃ is alkyl or aryl;

R₄, R₅ and R₆ ate independently selected from hydrogen, alkyl, aryl, halogen, hydroxy, nitro, cyano, alkoxy, aryloxy, CO₂R₇, COR₇, OCOR₇, SO₂R₇, SOR₇, SR₇, SO₂NR₇R₈CONR₇R₈, CONR₇NR₈R₉, OCONR₇R₈, NR₇R₈, NR₇COR₈, NR₇CONR₈R₉, NR₇CO₂R₈, NR₇SO₂R₈, CR₇═NOR₈, NR₇CONR₈NR₉R₁₀, NR₇NR₈CO₂R₉, NR₇NR₈CONR₉R₁₀, SO₂NR₇NR₈R₉, NR₇SO₂NR₈R₉, NR₇NR₈SO₂R₉, NR₇NR₈CO₂R₉, NR₇NR₈R₉ and NR₇CSNR₈R₉, or R₅ and R₆ together form a 5, 6 or 7 membered carbocyclic or heterocyclic ring; and

R₇, R₈, R₉, R₁₀, R₁₁ and R₁₂ are independently selected from hydrogen, alkyl and aryl, or a pharmaceutically acceptable salt or prodrug thereof.

U.S. Pat. No. 5,484,920 discloses useful adenosine A₂ₐ receptor antagonist compounds having the structural formula X 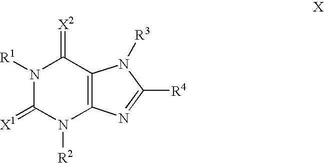
or a pharmaceutically acceptable salt thereof, wherein
R¹, R² and R³ are independently H, lower alkyl, lower alkenyl or lower alkynyl;
R⁴ is cycloalkyl, —(CH₂)ₙ—R⁵ or 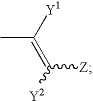
n is 0, 1, 2, 3, or 4;
R⁵ is optionally substituted aryl or optionally substituted heterocyclic;
Y¹ and Y² are independently H, halogen or lower alkyl;
Z is optionally substituted aryl, optionally substituted heterocyclic or 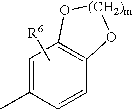
R⁶ is H, OH, lower alkyl, lower alkoxy, halogen, nitro or amino;
m is 1, 2, or 3; and
X¹ and X² are independently O or S.

Preferred compounds of formula X are those wherein R¹ and R² are methyl or ethyl; R³ is H or lower alkyl; R⁴ is 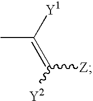
Y¹ and Y² are each H; X¹ and X² are each O; and Z is optionally substituted aryl of the formula 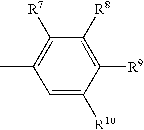
wherein at least one of R⁷, R⁸ and R⁹ is lower alkyl or lower alkoxy and the other are H, and R¹⁰ is H or lower alkyl, or Z is 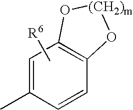
wherein R⁶ and m are as defined above.

Also preferred compounds of formula X are those wherein R¹ and R² are independently H, propyl, butyl, lower alkenyl or lower alkynyl; R⁴ is 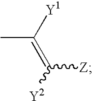
X¹ and X² are each O; Z is optionally substituted naphthyl or 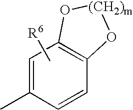
wherein R⁶, m, R³, Y¹ and Y² are as defined above.
Other A₂ₐ antagonists contemplated for use in the invention include:
piperazine-susbtituted triazolo[1,5-c]pyrimidines disclosed in U.S. Pat. No. 6,545,000;
triazolo[1,5-c]pyrimidines disclosed in U.S. Pat. No. 6,222,035;
xanthine derivatives disclosed in U.S. Pat. No. 5,703,085;
xanthine derivatives disclosed in U.S. Pat. No. 5,756,735;
thiazole derivatives disclosed in WO 2005/063743 having the formula XI 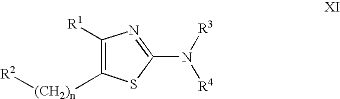
wherein n is 0, 1, 2, or 3;
R¹ is optionally substituted cycloalkyl, aryl, alicyclic heterocyclic or heteroaryl; R² includes halogen, optionally substituted alkyl, aryl, alicyclic heterocyclic, heteroaryl and —COR⁸; and
R³ and R⁴ independently include H, optionally substituted alkyl, optionally substituted aralkyl, and —COR¹².
2-aminoquinazoline derivatives disclosed in JP2005154434;
triazolo[1,5-c]pyrimidines disclosed in EP 1544200;
2-aminoquinoline derivatives disclosed in JP 2005132834;
triazolo[1,5-c]pyrimidines disclosed in WO 2003/068776;
triazolo[1,5-a]pyrimidines disclosed in WO 2003/020723
triazolo[1,5-a]pyrimidines disclosed in WO 1999/43678
pyrrolo[2,3-d]pyrimidines disclosed in US 2004/0092537;
thieno- and furano-pyrimidines disclosed in US 2004/0097524;
triazolo-pyrimidines disclosed in US 2004/0097526;
purine derivatives disclosed in US 2004/0102459;
pyrazolo[3,4-d]pyrimidines disclosed in US 2004/0116447;
pyrimidine compounds disclosed in WO 2005/079800 having the formula XII 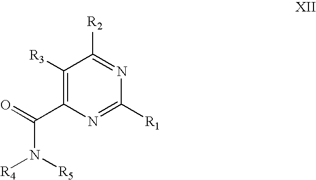
or a pharmaceutically acceptable salt thereof, wherein:
R₁ is optionally substituted alkyl, alkenyl or alkynyl, or —NR₆R₇, —OR₈, —SR₉ or halogen;
R₂ is optionally substituted aryl or heteroaryl attached via a carbon atom;
R₃ is H; optionally substituted alkyl, alkenyl, alkynyl or cycloalkyl; halogen; OH; or —OR₁₀;
R₄ is H; optionally substituted alkyl, alkenyl, alkynyl, cycloalkyl, aryl or heteroaryl;
R₅ is H or optionally substituted alkyl, alkenyl, alkynyl or cycloalkyl; or R₄ and R₅ together form a 5- or 6-membered heterocyclic ring;
R₆ is H or optionally substituted alkyl, alkenyl, alkynyl or cycloalkyl;
R₇, R₈, R₉ and R₁₀ are optionally substituted alkyl, alkenyl, alkynyl or cycloalkyl; or R₆ and R₇ together a 5- or 6-membered heterocyclic ring;
pyrimidine compounds disclosed in WO 2005/079801 having the formula XIII 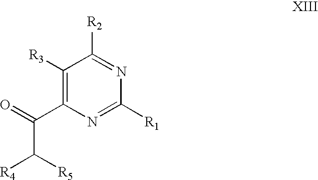
or a pharmaceutically acceptable salt thereof, wherein:
R₁ is H or —NH₂;
R₂ is optionally substituted aryl or heteroaryl attached via a carbon atom;
R₃ is H; optionally substituted alkyl, alkenyl, alkynyl or cycloalkyl; halogen; OH; or —OR₁₀;
R₄ is H; optionally substituted alkyl, alkenyl, alkynyl, cycloalkyl, aryl or heteroaryl;
R₅ is H or optionally substituted alkyl, alkenyl, alkynyl or cycloalkyl; or R₄ and R₅ together form a 5- or 6-membered heterocyclic ring;
R₁₀ is optionally substituted alkyl;
thiazolopyridines disclosed in US 2005/0065151;
2-acylaminobenzothiazole derivatives disclosed in U.S. Pat. No. 6,872,833;
benzoxazole derivatives disclosed in US 2004/0152702;
carboxamidobenzothiazole derivatives disclosed in U.S. Pat. No. 6,734,179;
7-phenyl-benzo[b]thiophen amide derivatives disclosed in U.S. Pat. No. 6,730,670;
7-amino-carboxamidobenzothiazole derivatives disclosed in U.S. Pat. No. 6,713,499;
7-heterocycle-substituted benzothiazole amide derivatives disclosed in U.S. Pat. No. 6,727,247;
2-(substituted aroylamino)-7-morpholinyl-benzothiazole derivatives disclosed in U.S. Pat. No. 6,624,163;
2-aminoacyl-7-morpholinyl-benzothiazole derivatives disclosed in U.S. Pat. No. 6,596,718;
nicotin- and isonicotinamide derivatives of benzothiazole disclosed in U.S. Pat. No. 6,620,811;
2-(pyridone-acylamino)-7-morpholinyl-benzothiazole derivatives disclosed in U.S. Pat. No. 6,599,901;
5-methoxy-8-aryl-triazolo[1,5-a]pyridine derivatives disclosed in U.S. Pat. No. 6,693,116;
8-amino-triazolo[1,5-a]pyridine-6-carboxylic acid amides disclosed in U.S. Pat. No. 6,689,790;
5-(phenyl or thiophenyl)-triazolo[1,5-a]pyridine derivatives disclosed in U.S. Pat. No. 6,514,989;
7-substituted 5-aminotriazolo[1,5-a]pyridine derivatives disclosed in U.S. Pat. No. 6,506,772;
2-(substituted amino)-benzothiazole derivatives disclosed in U.S. Pat. No. 6,521,754;
pyrimidinamines and pyridinamines disclosed in U.S. Pat. No. 6,586,441;
5-amino-substituted triazolo[1,5-a]pyridine derivatives disclosed in U.S. Pat. No. 6,355,653;
2,6-bis-heteroaryl-4-aminopyrimidines disclosed in WO 2005/058883;
4-pyrrolopyrimidinyl-benzenesulphonamide derivatives disclosed in WO 2003/082873;
benzofuran derivatives disclosed in WO2005/073210;
benzofuran derivatives disclosed in JP 2005126374;
N-thiazolylbenzamide derivatives disclosed in WO 2005/039572;
triazolopyrazine derivatives disclosed in WO 2004/092177;
triazolotriazines and derivatives thereof disclosed in WO 2004/092173;
triazolo- and pyrazolo-[1,5-c]pyrimidine derivatives disclosed in WO 2004/092172;
triazolo- and pyrazolo-[1,5-c]pyrimidine derivatives disclosed in WO 2004/092171; and
triazolotriazine and pyrazolotriazine derivatives disclosed in WO 2004/092179.

The US patents and applications cited herein are incorporated herein by reference. The adenosine A₂ₐ receptor antagonists are prepared by known methods as described in the cited patents and applications.

As used herein, “patient” means a mammal, especially a human.

It is contemplated that more than one adenosine A₂ₐ receptor antagonist (e.g., 2 or 3) can be administered to treat EPS, dystonia, RLS or PLMS; preferably, one adenosine A₂ₐ receptor antagonist is administered.

Antipsychotic agents causing the EPS treated by adenosine A₂ₐ receptor antagonists and for use in combination with adenosine A₂ₐ receptor antagonists include typical and atypical antipsychotic agents. Typical antipsychotics include loxapine, haloperidol, chlorpromazine, prochlorperazine and thiothixene. Atypical antipsychotics include clozapine, olanzapine, loxapine, quetiapine, ziprasidone, risperidone, aripiprazole, sertindole and zotepine.

Tricyclic antidepressants causing dystonia treated by adenosine A₂ₐ receptor antagonists include perphenazine, amitriptyline, desipramine, doxepin, trimipramine and protriptyline. Anticonvulsants which may cause dystonia, but which also may be useful in treating RLS or PLMS include phenytoin, carbamazepine and gabapentin.

Dopamine agonists useful in treating RLS and PLMS include pergolide, pramipexole, ropinerole, fenoldopam and cabergoline.

Opioids useful in treating PRLS and PLMS include codeine, hydrocodone, oxycodone, propoxyphene and tramadol.

Benzodiazepines useful in treating PRLS and PLMS include clonazepam, triazolam and temazepam.

The antipsychotics, tricyclic antidepressants, anticonvulsants, dopamine agonists, opioids and benzodiazepines are commercially available and are described in the literature, e.g., in The Physicians' Desk Reference (Montvale: Medical Economics Co., Inc., 2001).

It is contemplated that two or more A₂ₐ receptor antagonists could be administered in combination with one or more other agents (e.g., antipsychotics, tricyclic antidepressants, anticonvulsants, dopamine agonists, opioids or benzodiazepines), although administration of one A₂ₐ antagonist in combination with one other agent is preferred for each of the indications.

Administration of separate dosage forms of the A₂ₐ antagonist(s) and the other agent(s) are one preferred embodiment. Another preferred embodiment is fixed-dose pharmaceutical compositions, i.e., single dosage forms consisting of the A₂ₐ receptor antagonist(s) in combination with the other agent(s) for the treatment or prevention of EPS, dystonia, RLS or PLMS and a pharmaceutically acceptable carrier. Preferred fixed-dose compositions consist of one A₂ₐ receptor antagonist and one other agent for treating or preventing EPS, dystonia, RLS or PLMS and a pharmaceutically acceptable carrier.

Preferred adenosine A₂ₐ antagonists are those described in U.S. Pat. No. 6,630,475.

A particularly preferred compound of the invention is Compound A of the formula 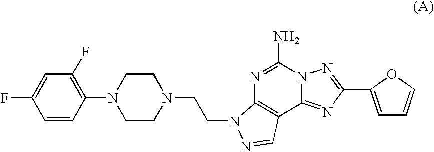
or a pharmaceutically acceptable salt or solvate thereof, disclosed in U.S. Pat. No. 6,630,475 and listed as the first compound in the table of compounds of structure 1.

Another preferred compound is Compound B of the formula 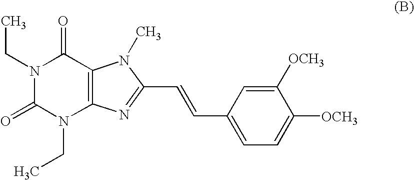
or a pharmaceutically acceptable salt or solvate thereof, disclosed in U.S. Pat. No. 5,484,920 and known as istradefylline.

Compounds useful in the method of the invention will show utility as adenosine A₂ₐ receptor antagonists in these assays.

Human Adenosine A₂ₐ and A₁ Receptor Competition Binding Assay Protocol Membrane sources: A₂ₐ: Human A₂ₐ Adenosine Receptor membranes, Catalog #RB-HA2a, Receptor Biology, Inc., Beltsville, Md. Dilute to 17 μg/100 μl in membrane dilution buffer (see below).

Assay Buffers: Membrane dilution buffer: Dulbecco's Phosphate Buffered Saline (Gibco/BRL)+10 mM MgCl₂.

Compound Dilution Buffer: Dulbecco's Phosphate Buffered Saline (Gibco/BRL)+10 mM MgCl₂ supplemented with 1.6 mg/ml methyl cellulose and 16% DMSO. Prepared fresh daily.

Ligands: A₂ₐ: [3H]—SCH 58261, custom synthesis, AmershamPharmacia Biotech, Piscataway, N.J. Stock is prepared at 1 nM in membrane dilution buffer. Final assay concentration is 0.5 nM.

A₁: [3H]-DPCPX, AmershamPharmacia Biotech, Piscataway, N.J. Stock is prepared at 2 nM in membrane dilution buffer. Final assay concentration is 1 nM.

Non-Specific Binding:

A₂ₐ: To determine non-specific binding, add 100 nM CGS 15923 (RBI, Natick, Mass.). Working stock is prepared at 400 nM in compound dilution buffer.

A₁: To determine non-specific binding, add 100 μM NECA (RBI, Natick, Mass.). Working stock is prepared at 400 μM in compound dilution buffer.

Compound Dilution:

Prepare 1 mM stock solutions of compounds in 100% DMSO. Dilute in compound dilution buffer. Test at 10 concentrations ranging from 3 μM to 30 pM. Prepare working solutions at 4× final concentration in compound dilution buffer.

Assay Procedure:

Perform assays in deep well 96 well plates. Total assay volume is 200 μl. Add 50 μl compound dilution buffer (total ligand binding) or 50 μl CGS 15923 working solution (A₂ₐ non-specific binding) or 50 μl NECA working solution (A₁ non-specific binding) or 50 μl of drug working solution. Add 50 μl ligand stock ([3H]—SCH 58261 for A₂ₐ, [3H]-DPCPX for A₁). Add 100 μl of diluted membranes containing the appropriate receptor. Mix. Incubate at room temperature for 90 minutes. Harvest using a Brandel cell harvester onto Packard GF/B filter plates. Add 45 μl Microscint 20 (Packard), and count using the Packard TopCount Microscintillation Counter. Determine IC₅₀ values by fitting the displacement curves using an iterative curve fitting program (Excel). Determine Ki values using the Cheng-Prusoff equation.

Haloperidol-Induced Catalepsy in the Rat

Male Sprague-Dawley rats (Charles River, Calco, Italy) weighing 175-200 g are used. The cataleptic state is induced by the subcutaneous administration of the dopamine receptor antagonist haloperidol (1 mg/kg, sc), 90 min before testing the animals on the vertical grid test. For this test, the rats are placed on the wire mesh cover of a 25×43 plexiglas cage placed at an angle of about 70 degrees with the bench table. The rat is placed on the grid with all four legs abducted and extended (“frog posture”). The use of such an unnatural posture is essential for the specificity of this test for catalepsy. The time span from placement of the paws until the first complete removal of one paw (descent latency) is measured maximally for 120 sec.

The selective A_{2A} adenosine antagonists under evaluation are administered orally at doses ranging between 0.03 and 3 mg/kg, 1 and 4 h before scoring the animals.

In separate experiments, the anti-cataleptic effects are determined for the reference compound, L-DOPA (25, 50 and 100 mg/kg, ip),

The following example shows the use of adenosine A2a antagonists to attenuate the Extra-Pyramidal Syndrome (EPS) displayed in cebus apella monkeys sensitized to the dopamine D₂ receptor antagonist, haloperidol.

### EXAMPLE

A colony of seven Cebus apella monkeys that were previously sensitized to the chronic effects of haloperidol, exhibit EPS when administered haloperidol acutely (0.3 mg/kg, p.o.). Compound A was administered orally (p.o.) at doses of 0.3-30 mg/kg, in conjunction with haloperidol. Compound B was administered orally (p.o.) at doses of 3-100 mg/kg, in conjunction with haloperidol. The studies were conducted using a within-subjects design such that each monkey received all 6 treatments (vehicle and 5 doses of Compound A) in a crossover, balanced design. In all the studies, the group of seven monkeys exhibited baseline levels of EPS when dosed with haloperidol.

Compound A produced a dose-dependent reduction in the maximum EPS score (FIG. 1A), as well as a dose-dependent delay in the onset of EPS (FIG. 1B). At a dose of 1 mg/kg, Compound A prevented the onset of EPS in one monkey, and delayed the onset of EPS by 1 hr. Compound A, at a dose of 3 mg/kg, prevented the onset of EPS in two monkeys, and delayed the onset of EPS by almost 2 hr in the remaining monkeys. At 10 and 30 mg/kg, Compound A prevented the onset of EPS in three monkeys and delayed the onset of EPS by an average of 2.3-2.9 hr.

Compound B produced a reduction in the maximum EPS score (FIG. 2A), as well as a dose-dependent delay in the onset of EPS (FIG. 2B). Additionally, Compound B prevented the onset of EPS in one monkey at 3-30 mg/kg, and in two monkeys at doses of 57 and 100 mg/kg.

Clinical guidelines for the treatment of RLS and PLMS have been established: see A. L. Chesson et al, Sleep, 22, 7 (1999), p. 961-8. Efficacy of adenosine A₂ₐ antagonists in treating RLS and PLMS can be determined by a method analogous to the clinical method described in the literature for pramipexole and ropinerole by Weimerskirch et al, Annals of Pharmacotherapy, 35, 5 (2001), p. 627-30.

For preparing pharmaceutical compositions from the compounds useful in the method of this invention, inert, pharmaceutically acceptable carriers can be either solid or liquid. Solid form preparations include powders, tablets, dispersible granules, capsules, cachets and suppositories. The powders and tablets may be comprised of from about 0.1 to about 99 percent active ingredient. Suitable solid carriers are known in the art, e.g. magnesium carbonate, magnesium stearate, talc, sugar, lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

The compounds useful in the method of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the adenosine A₂ₐ receptor antagonist and the antipsychotic are administered orally.

Preferably, the pharmaceutical preparation is in unit dosage form. In such form, the preparation is subdivided into unit doses containing appropriate quantities of the active component, e.g., an effective amount to achieve the desired purpose.

The quantity of adenosine A₂ₐ receptor antagonist in a unit dose of preparation may be varied or adjusted from about 0.1 mg to 1000 mg, more preferably from about 1 mg to 300 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day if desired.

The amount and frequency of administration of the adenosine A₂ₐ receptor antagonist useful in the method of the invention will be regulated according to the judgment of the attending clinician considering such factors as age, condition and size of the patient as well as severity of the symptoms being treated. A typical recommended dosage regimen for an adenosine A₂ₐ receptor antagonist is oral administration of about 10 mg to 2000 mg/day preferably 10 to 1000 mg/day, in two to four divided doses to provide relief from the effects of EPS, dystonia, RLS or PLMS. The compounds are non-toxic when administered within this dosage range.

The doses and dosage regimen of the other agents used in combination with the adenosine A₂ₐ receptor antagonists, i.e., the antipsychotics, tricyclcic antidepressants, anticonvulsants, dopamine agonists, benzodiazepines, opioids, lithium or iron, will be determined by the attending clinician in view of the approved doses and dosage regimen in the package insert, taking into consideration the age, sex and condition of the patient and the severity of the disease. When administered in combination, the adenosine A₂ₐ receptor antagonist and the other agent can be administered simultaneously or sequentially. This is particularly useful when the components of the combination are preferably given on different dosing schedules, e.g., one component is administered daily and the other every six hours, or when the preferred pharmaceutical compositions are different, e.g. one is preferably a tablet and one is a capsule. It is therefore advantageous to provide the adenosine A₂ₐ receptor antagonist and the other agent in a kit comprising, in separate containers in a single package, pharmaceutical compositions for use in combination to treat or prevent EPS, dystonia, RLS or PLMS, wherein one container comprises a pharmaceutical composition comprising an effective amount of an adenosine A₂ₐ receptor antagonist in a pharmaceutically acceptable carrier, and wherein a separate container comprises a pharmaceutical composition comprising an effective amount of another agent appropriate to treat the indicated condition.

Those skilled in the art will recognize that a dosage form for one of the components of the combination can be modified to contain both an adenosine A₂ₐ receptor antagonist and another agent, e.g., an adenosine A₂ₐ receptor antagonist and an antipsychotic or an adenosine A₂ₐ receptor antagonist and a dopamine agonist.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

We claim:

1. A pharmaceutical composition consisting of a therapeutically effective amount of a combination of an adenosine A₂ₐ receptor antagonist and an antipsychotic agent and a pharmaceutically acceptable carrier.

2. The composition of claim 1 wherein the antipsychotic agent is a typical antipsychotic agent selected from the group consisting of loxapine, haloperidol, chlorpromazine, prochlorperazine and thiothixene, or an atypical antipsychotic agent selected from the group consisting of clozapine, olanzapine, loxapine, quetiapine, ziprasidone, risperidone, aripiprazole, sertindole or zotepine.

3. The composition of claim 2 wherein the adenosine A₂ₐ receptor antagonist is selected from the group consisting of compounds of formula I 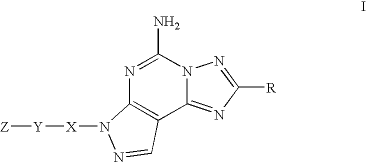
or a pharmaceutically acceptable salt thereof, wherein
R is R¹-furanyl, R¹-thienyl, R¹-pyridyl, R¹-pyridyl N-oxide, R¹-oxazolyl, R¹⁰-phenyl, R¹-pyrrolyl or C₄-C₆ cycloalkenyl;
X is C₂-C₆ alkylene or —C(O)CH₂—;
Y is —N(R²)CH₂CH₂N(R³)—, —OCH₂CH₂N(R²)—, —O—, —S—, —CH₂S—, —(CH₂)₂—NH—, or 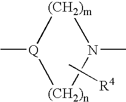
and
Z is R⁵-phenyl, R⁵-phenyl(C₁-C₆)alkyl, R⁵-heteroaryl, diphenylmethyl, R⁶—C(O)—, R⁶—SO₂—, R⁶—OC(O)—, R⁷—N(R⁸)—C(O)—, R⁷—N(R⁸)—C(S)—, 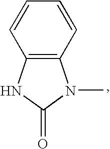
phenyl-CH(OH)—, or phenyl-C(═NOR²)—; or when Q is 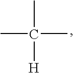
Z is also phenylamino or pyridylamino; or
Z and Y together are 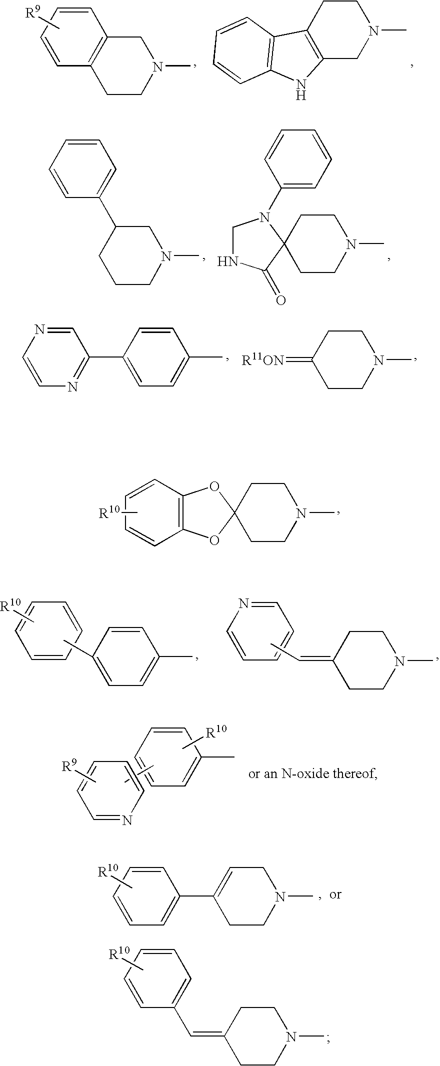
R¹ is 1 to 3 substituents independently selected from hydrogen, C₁-C₆-alkyl, —CF₃, halogen, —NO₂, —NR¹²R¹³, C₁-C₆ alkoxy, C₁-C₆ alkylthio, C₁-C₆ alkylsulfinyl, and C₁-C₆ alkylsulfonyl;
R² and R³ are independently selected from the group consisting of hydrogen and C₁-C₆ alkyl;
m and n are independently 2-3;
Q is 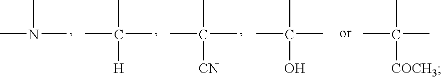
R⁴ is 1-2 substituents independently selected from the group consisting of hydrogen and C₁-C₆alkyl, or two R⁴ substituents on the same carbon can form ═O;
R⁵ is 1 to 5 substituents independently selected from the group consisting of hydrogen, halogen, C₁-C₆ alkyl, hydroxy, C₁-C₆ alkoxy, —CN, di-((C₁-C₆)alkyl)amino, —CF₃, —OCF₃, acetyl, —NO₂, hydroxy(C₁-C₆)alkoxy, (C₁-C₆)-alkoxy(C₁-C₆)alkoxy, di-((C₁-C₆)-alkoxy)(C₁-C₆)alkoxy, (C₁-C₆)-alkoxy(C₁-C₆)alkoxy-(C₁-C₆)-alkoxy, carboxy(C₁-C₆)-alkoxy, (C₁-C₆)-alkoxycarbonyl(C₁-C₆)alkoxy, (C₃-C₆)cycloalkyl(C₁-C₆)alkoxy, di-((C₁-C₆)alkyl)amino(C₁-C₆)alkoxy, morpholinyl, (C₁-C₆)alkyl-SO₂—, (C₁-C₆)alkyl-SO—(C₁-C₆)alkoxy, tetrahydropyranyloxy, (C₁-C₆)alkylcarbonyl(C₁-C₆)-alkoxy, (C₁-C₆)-alkoxycarbonyl, (C₁-C₆)alkylcarbonyloxy(C₁-C₆)-alkoxy, —SO₂NH₂, phenoxy, 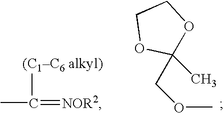
or adjacent R⁵ substituents together are —O—CH₂—O—, —O—CH₂CH₂—O—, —O—CF₂—O— or —O—CF₂CF₂—O— and form a ring with the carbon atoms to which they are attached;
R⁶ is (C₁-C₆)alkyl, R⁵-phenyl, R⁵-phenyl(C₁-C₆)alkyl, thienyl, pyridyl, (C₃-C₆)cycloalkyl, (C₁-C₆)alkyl-OC(O)—NH—(C₁-C₆)alkyl-, di-((C₁-C₆)alkyl)aminomethyl, or 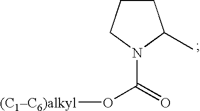
R⁷ is (C₁-C₆)alkyl, R⁵-phenyl or R⁵-phenyl(C₁-C₆)alkyl;
R⁸ is hydrogen or C₁-C₆ alkyl; or R⁷ and R⁸ together are —(CH₂)ₚ-A-(CH₂)_{q}, wherein p and q are independently 2 or 3 and A is a bond, —CH₂—, —S— or —O—, and form a ring with the nitrogen to which they are attached;
R⁹ is 1-2 groups independently selected from hydrogen, C₁-C₆ alkyl, hydroxy, C₁-C₆ alkoxy, halogen, —CF₃ and (C₁-C₆)alkoxy(C₁-C₆)alkoxy;
R¹⁰ is 1 to 5 substituents independently selected from the group consisting of hydrogen, halogen, C₁-C₆ alkyl, hydroxy, C₁-C₆ alkoxy, —CN, —NH₂, C₁-C₆alkylamino, di-((C₁-C₆)alkyl)amino, —CF₃, —OCF₃ and —S(O)₀₋₂(C₁-C₆)alkyl;
R¹¹ is H, C₁-C₆ alkyl, phenyl, benzyl, C₂-C₆ alkenyl, C₁-C₆ alkoxy(C₁-C₆)alkyl, di-((C₁-C₆)alkyl)amino(C₁-C₆)alkyl, pyrrolidinyl(C₁-C₆)alkyl or piperidino(C₁-C₆)alkyl;
R¹² is H or C₁-C₆ alkyl; and
R¹³ is (C₁-C₆)alkyl-C(O)— or (C₁-C₆)alkyl-SO₂—;
and compounds of formula X 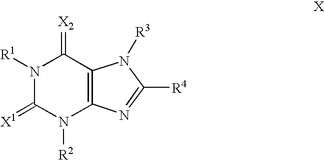
or a pharmaceutically acceptable salt thereof, wherein
R¹, R² and R³ are independently H, lower alkyl, lower alkenyl or lower alkynyl;
R⁴ is cycloalkyl, —(CH₂)ₙ—R⁵ or 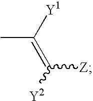
n is 0, 1, 2, 3, or 4;
R⁵ is optionally substituted aryl or optionally substituted heterocyclic;
Y¹ and Y² are independently H, halogen or lower alkyl;
Z is optionally substituted aryl, optionally substituted heterocyclic or 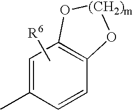
R⁶ is H, OH, lower alkyl, lower alkoxy, halogen, nitro or amino;
m is 1, 2, or 3; and
X¹ and X² are independently O or S.

4. The composition of claim 3 wherein the adenosine A₂ₐ receptor antagonist is 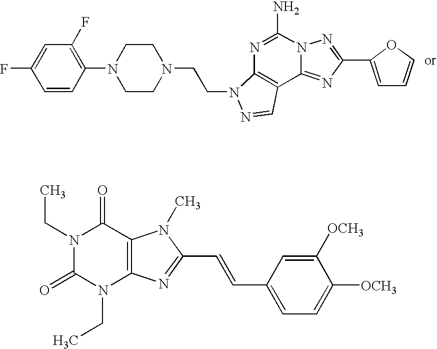
or a pharmaceutically acceptable salt or solvate thereof.

5. The composition of claim 2 wherein the adenosine A₂ₐ receptor antagonist is 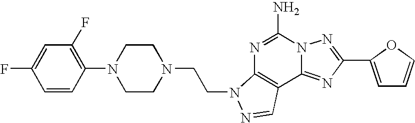
or a pharmaceutically acceptable salt or solvate thereof.

6. The composition of claim 2 wherein the adenosine A₂ₐ receptor antagonist is 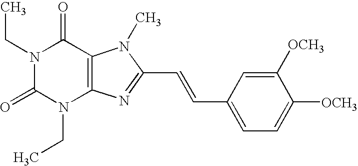
or a pharmaceutically acceptable salt or solvate thereof.

7. A pharmaceutical composition consisting of a therapeutically effective amount of a combination of an adenosine A₂ₐ receptor antagonist and an anticonvulsant and a pharmaceutically acceptable carrier.

8. The composition of claim 7 wherein the anticonvulsant is selected from the group consisting of phenytoin, carbamazepine and gabapentin.

9. The composition of claim 8 wherein the adenosine A₂ₐ receptor antagonist is 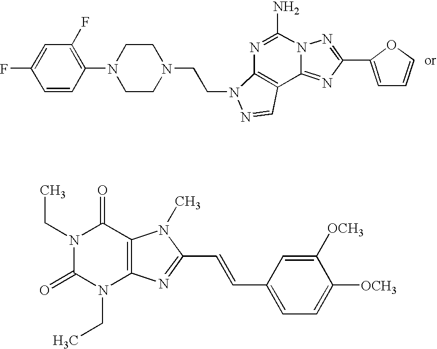
or a pharmaceutically acceptable salt or solvate thereof.

10. A pharmaceutical composition consisting of a therapeutically effective amount of a combination of an adenosine A₂ₐ receptor antagonist and lithium and a pharmaceutically acceptable carrier.

11. The composition of claim 10 wherein the adenosine A₂ₐ receptor antagonist is 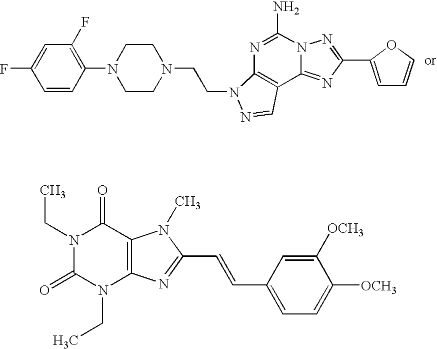
or a pharmaceutically acceptable salt or solvate thereof.

12. A pharmaceutical composition consisting of a therapeutically effective amount of a combination of an adenosine A₂ₐ receptor antagonist and an opioid and a pharmaceutically acceptable carrier.

13. The composition of claim 12 wherein the opioid is selected from the group consisting of codeine, hydrocodone, oxycodone, propoxyphene and tramadol.

14. The composition of claim 13 wherein the adenosine A₂ₐ receptor antagonist is 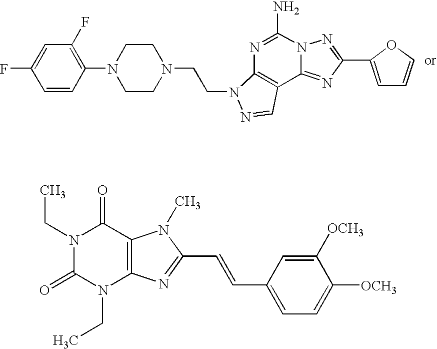
or a pharmaceutically acceptable salt or solvate thereof.

15. A method of treating or preventing Extra-Pyramidal Syndrome wherein the Extra-Pyramidal Syndrome has been caused by treatment with sertindole or zotepine comprising administering to a patient in need thereof a therapeutically effective amount of an adenosine A₂ₐ receptor antagonist selected from the group consisting of 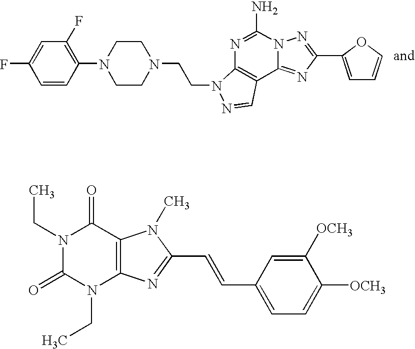
or a pharmaceutically acceptable salt or solvate thereof.
